(19) United States
(12) Patent Application Publication
(10) Pub. No.: US 2011/0065715 A1
(43) Pub. Date: Mar. 17, 2011

(54) Nogo Receptor Binding Small Molecules to Promote Axonal Growth

(75) Inventors: Stephen M. Strittmatter, Guilford, CT (US); Erik Gunther, Branford, CT (US)

(73) Assignee: Yale University, New Haven, CT (US)

(21) Appl. No.: 12/745,150

(22) PCT Filed: Nov. 26, 2008
(86) PCT No.: PCT/US08/13178

§ 371 Date: Nov. 23, 2010

Related U.S. Application Data
(60) Provisional application No. 60/990,686, filed on Nov. 28, 2007.

(51) Int. Cl. A61K 31/4985; G01N 33/53; C40B 30/04; A61K 31/275; C12N 5/079; A61K 31/519; A61K 31/343; A61K 31/47; A61K 31/428; A61K 31/404; A61K 31/35; A61K 31/135; A61P 25/00

(52) U.S. Cl. 514/250; 436/501; 506/9; 514/520; 435/375; 514/260.1; 514/469; 514/311; 514/367; 514/415; 514/456; 514/646

(57) ABSTRACT

The present invention provides a method for identifying compounds which modulate the interaction of Nogo and Nogo receptor (NgR). The present invention also provides compounds that modulate the interaction of Nogo and Nogo receptor (NgR), the use of such compounds and compositions in the treatment or amelioration of conditions diseases or disorders, such as spinal cord injury, traumatic brain injury, stroke, multiple sclerosis, ALS, Huntington's disease, Alzheimer's disease, Parkinson's disease, epilepsy, Schizophrenia or schizoaffective disorders.

### FIELD OF THE INVENTION

This invention relates to Nogo receptor antagonists and agonists. More particularly, the invention relates to compounds that modulate neuronal axonal growth.

### BACKGROUND OF THE INVENTION

Axons and dendrites of neurons are long cellular extensions from neurons. The distal tip of an extending axon or neurite comprises a specialized region, known as the growth cone. Growth cones sense the local environment and guide axonal growth toward the neuron's target cell. Growth cones respond to several environmental cues, for example, surface adhesiveness, growth factors, neurotransmitters and electric fields. The guidance of growth at the cone involves various classes of adhesion molecules, intercellular signals, as well as factors that stimulate and inhibit growth cones. The growth cone of a growing neurite advances at various rates, but typically at the speed of one to two millimeters per day.

Growth cones are hand shaped, with broad flat expansion (microspikes or filopodia) that differentially adhere to surfaces in the embryo. The filopodia are continually active, some filopodia retract back into the growth cone, while others continue to elongate through the substratum. The elongations between different filopodia form lamellipodia.

The growth cone explores the area that is ahead of it and on either side with its lamellipodia and filopodia. When an elongation contacts a surface that is unfavorable to growth, it withdraws. When an elongation contacts a favorable growth surface, it continues to extend and guides the growth cone in that direction. The growth cone can be guided by small variations in surface properties of the substrata. When the growth cone reaches an appropriate target cell a synaptic connection is created.

Nerve cell function is greatly influenced by the contact between the neuron and other cells in its immediate environment (U. Rutishauser, T. M. Jessell, Physiol. Rev. 68:819 (1988)). These cells include specialized glial cells, oligodendrocytes in the central nervous system (CNS), and Schwann cells in the peripheral nervous system (PNS), which ensheathe the neuronal axon with myelin (an insulating structure of multi-layered membranes) (G. Lemke, in An Introduction to Molecular Neurobiology, Z. Hall, Ed. (Sinauer, Sunderland, Mass.), p. 281 (1992)).

While CNS neurons have the capacity to regenerate after injury, they are inhibited from doing so because of the presence of inhibitory proteins present in myelin and possibly also by other types of molecules normally found in their local environment (Brittis and Flanagan, Neuron 30:11-14 (2001); Jones et al., J. Neurosc. 22:2792-2803 (2002); Grimpe et al., J. Neurosci. 22:3144-3160 (2002)).

Several myelin inhibitory proteins that are found on oligodendrocytes have been characterized, e.g., NogoA (Chen et al., Nature 403:434-439 (2000); Grandpre et al., Nature 403:439-444 (2000)), myelin associated glycoprotein (MAG, McKerracher et al., Neuron 13:805-811 (1994); Mukhopadhyay et al., Neuron 13:757-767 (1994)) and oligodendrocyte glycoprotein (OM-gp, Mikol and Stefansson, J. Cell. Biol. 106:1273-1279 (1988)). Each of these proteins has been separately shown to be a ligand for the neuronal Nogo receptor-1 (Wang et al., Nature 417:941-944 (2002); Liu et al., Science 297:1190-93 (2002); Grandpre et al., Nature 403:439-444 (2000); Chen et al., Nature 403:434-439 (2000); Domeniconi et al., Neuron 35:283-90 (2002)).

Nogo receptor-1 is a GPI-anchored membrane protein that contains 8 leucine rich repeats (Fournier et al., Nature 409:341-346 (2001)). Upon interaction with an inhibitory protein (e.g., NogoA, MAG and OM-gp), the Nogo receptor-1 complex transduces signals that lead to growth cone collapse and inhibition of neurite outgrowth.

There is an urgent need for molecules that inhibit Nogo receptor-1 binding to its ligands, stimulate neurite outgrowth and attenuate myelin-mediated growth cone collapse and inhibition of neurite outgrowth.

### SUMMARY OF THE INVENTION

The present invention includes a method for identifying compounds which modulate the interaction of Nogo and Nogo receptor (NgR). The method include: (a) mixing a Nogo polypeptide, a NgR polypeptide and a test compound; (b) measuring an interference of the binding of said Nogo polypeptide to said NgR polypeptide in the presence of said compound, as compared to the binding of said Nogo polypeptide to said NgR polypeptide in the absence of said compound. In some embodiments, the Nogo polypeptide is Nogo-66 polypeptide, and NgR polypeptide is Fc-NgR polypeptide.

In some embodiments, the interference is measured by light signal emitted from a complex which is formed between a donor bead and a receptor bead. The NgR polypeptide binds to a biomolecule which is conjugated with the donor bead. The Nogo polypeptide binds to a biomolecule which is conjugated with the receptor bead. The donor bead contains a photosensitizer, and the receptor bead contains a chemiluminescer.

In some embodiments, where the interference is detected, the interference is further confirmed by detecting an intrinsic interference of the compound by a dose-response assay. The assay includes: (a) incubating donor beads, receptor beads and the compound at different concentrations; and (b) measuring the intrinsic interference of the compound at different concentrations by light signal emitted from a complex which is formed between the donor bead and the receptor bead. The donor bead is conjugated with a biomolecule and contains a photosensitizer. The receptor bead is conjugated with a biomolecule and contains a chemiluminescer. In one embodiment, the assay can be conducted using AlphaScreen TruHits™ Kit (PerkinElmer).

In some embodiments, the method is conducted in a multi-well plate with a plurality of test compounds. In some embodiments, the test compound is a member of a diverse small molecule library. In some embodiments, such small molecule library contains 20,000 compounds. The method includes screening the entire library or any subset thereof.

In some embodiments, the test compound is a member of a small molecule library consisting of drug-like organic compounds which have molecular weight of no more than 500 daltons, and have no more than 5 nitrogen or 5 oxygen atoms. The method includes screening the entire library or any subset thereof.

In some embodiments, the test compound is a member of a focused small molecule library consisting of compounds which are structurally similar or related to compounds which are previously identified to modulate the interaction of Nogo and Nogo receptor.

The present invention includes a method for identifying compounds which inhibit the interaction of Nogo and Nogo receptor (NgR), i.e., Nogo receptor-1 antagonists.

The present invention also includes a method for identifying compounds which enhance the interaction of Nogo and Nogo receptor (NgR), i.e., Nogo receptor-1 agonists.

The present invention includes a method of identifying compounds which promote neurite outgrowth. The method includes: (a) screening a small molecule library for compounds which interfere with the interaction of Nogo and Nogo receptor (NgR) as described above, and (b) isolating a candidate compound.

In some embodiments, the method further includes conducting a secondary dose-response assay of the candidate compound. In some embodiments, the secondary dose-response assay is Enzyme-Linked ImmunoSorbent Assay (ELISA) or Dissociation-Enhanced Lanthanide Fluorescent Immunoassay (DELFIA).

In some embodiments, the method further includes conducting a functional assay by measuring neurite outgrowth activity of the candidate compound, wherein the candidate compound promotes neurite outgrowth.

The present invention also includes a method of identifying compounds which inhibit neurite outgrowth. The method includes: (a) screening a small molecule library for compounds which interfere with the interaction of Nogo and Nogo receptor (NgR) as described above, and (b) isolating a candidate compound.

In some embodiments, the method further includes conducting a secondary dose-response assay of the candidate compound, such as Enzyme-Linked ImmunoSorbent Assay (ELISA) or Dissociation-Enhanced Lanthanide Fluorescent Immunoassay (DELFIA).

In some embodiments, the method further includes conducting a functional assay by measuring neurite outgrowth activity of the candidate compound, wherein the candidate compound inhibit neurite outgrowth.

The present invention provides compounds that modulate the interaction of Nogo and Nogo receptor (NgR). Such compounds include an optionally substituted, optionally partially saturated benzofuran, indole, thiazolopyrimidine, pyrroloquinoxaline, benzothiazole, chromene or quinoline, having a molecular weight of no more than 500 daltons, and containing no more than 5 nitrogen or 5 oxygen atoms.

In some embodiments, the compounds can be an optionally substituted 5-hydroxy-benzofuran, or an optionally substituted 5-hydroxy-3-aroylalkylbenzofuran, having a molecular weight of no more than 500 daltons, and containing no more than 5 nitrogen or 5 oxygen atoms.

In some embodiments, the compounds can be an optionally substituted 3-acyl-indole, or an optionally substituted 3-hydroxy-3-aroylalkyl-1,3-dihydro-2H-indol-2-one, having a molecular weight of no more than 500 daltons, and containing no more than 5 nitrogen or 5 oxygen atoms.

The present invention further provides compounds that modulate the interaction of Nogo and Nogo receptor (NgR). The compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, N1,N1-dimethyl-4-[4-(dimethylamino)benzyl]aniline, 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-{2-[3-(2-methylprimidin-4-yl-phenyl]hydrazono}-3-oxopropanenitrile, or a pharmaceutically acceptable salt thereof.

The present invention includes the use of Nogo receptor-1 antagonists for promoting neurite outgrowth, neuronal survival, and axonal regeneration in neurons. The invention features compounds and methods useful for inhibiting neurite outgrowth inhibition, promoting neuronal survival, and/or promoting axonal regeneration in neurons. In some embodiments, the compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methylThenzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline, or a pharmaceutically acceptable salt thereof.

The present invention also includes the use of Nogo receptor-1 agonists for inhibiting neurite outgrowth. The invention features compounds and methods useful for inhibiting neurite outgrowth, inhibiting neuronal survival, and/or inhibiting axonal regeneration in neurons. In some embodiments, the compounds are 4-[(4-oxo-2-thioxo-1,3-thiazolan-3-yl)methyl]benzonitrile, 5-bromo-3-[2-(4-chlorophenyl)-2-oxoethyl]-3 -hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-{2-[3-(2-methylprimidin-4-yl-phenyl]hydrazono}-3-oxopropanenitrile, or a pharmaceutically acceptable salt thereof.

The present invention includes a method of promoting neurite outgrowth in neurons. The method includes contacting the neuron with an effective amount of a compound that promotes neurite outgrowth as described above. In some embodiments, compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl- 1 ,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3 -(4-chlorobenzoyl)-6-methyl-4H-chromen-4-one, and N1,N1-dimethyl-4-[4-(dimethylamino)benzyl]aniline, or a pharmaceutically acceptable salt thereof.

The present invention also includes a method of inhibiting signal transduction by the NgR1 signaling complex. The method includes contacting a neuron with an effective amount of a compound that promotes neurite outgrowth as described above. In some embodiments, the compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-1H-indol-1-yl)methyl]benzonitrile, 3-(4-chlorobenzoyl)-6-methyl-4H-chromen-4-one, and N1 ,N1 -dimethyl-4-[4-(dimethylamino)benzyl]aniline, or a pharmaceutically acceptable salt thereof.

The present invention includes a method of treating a central nervous system (CNS) disease or disorder. The method includes administering to a mammal, e.g., a human, an effective amount of a compound that promotes neurite outgrowth or axonal regeneration as described above. In some embodiments, the compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline, or a pharmaceutically acceptable salt thereof.

In some embodiments, the central nervous system (CNS) disease or disorder is a result of cranial or cerebral trauma, spinal cord injury, stroke or a demyelinating disease. Examples of CNS disease or disorder are multiple sclerosis, ALS, Huntington's disease, Alzheimer's disease, Parkinson's disease, diabetic neuropathy, stroke, traumatic brain injuries, spinal cord injury, optic neuritis, glaucoma, hearing loss, adrenal leukodystrophy, monophasic demyelination, encephalomyelitis, multifocal leukoencephalopathy, panencephalitis, Marchiafava-Bignami disease, pontine myelinolysis, adrenoleukodystrophy, Pelizaeus-Merzbacher disease, Spongy degeneration, Alexander's disease, Canavan's disease, metachromatic leukodystrophy, epilepsy and Krabbe's disease.

In some embodiments, the compound is administered by oral, parenteral, subcutaneous, intravenous, intramuscular, intraperitoneal, transdermal, intracranial or buccal administration.

The present invention further includes a method of promoting neurite outgrowth or axonal regeneration in neurons. The method includes contacting the neuron with an effective amount of a compound that promotes neurite outgrowth or axonal regeneration as described above. In some embodiments, the compounds are 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline, or a pharmaceutically acceptable salt thereof.

The present invention also includes a method of inhibiting neurite outgrowth or axonal regeneration in neurons. The method includes contacting the neuron with an effective amount of a compound that inhibits neurite outgrowth or axonal regeneration as described above. In some embodiments, the compounds are 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-{2-[3-(2-methylprimidin-4-yl-phenyl]hydrazono}-3-oxopropanenitrile, or a pharmaceutically acceptable salt thereof.

The present invention further includes a method of treating Schizophrenia or schizoaffective disorders. The method includes administering to a mammal, e.g., a human, an effective amount of a compound that inhibits neurite outgrowth or axonal regeneration as described above. In some embodiments, the compounds are 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3 -hydroxy-1,3-dihydro-2H-indol-2-one, and 3-(4-chlorophenyl)-2-{2-[3-(2-methylprimidin-4-yl-phenyl]hydrazono}-3-oxopropanenitrile, or a pharmaceutically acceptable salt thereof.

The present invention also includes a composition that contains a compound that promotes neurite outgrowth as described above, or a compound that inhibits neurite outgrowth as described above, and a pharmaceutically acceptable carrier.

### BRIEF DESCRIPTION OF THE DRAWINGS

FIG. 1 is a schematic illustration of an AlphaScreen assay. Streptavidin-acceptor bead and Protein A-donor bead in solution produce no signal by themselves.

FIG. 2 is schematic illustration of an AiphaScreen assay. Streptavidin-acceptor bead binds a biotinylated Ng66 and Protein A-donor bead binds a Fc-NgR fusion protein, bringing the Streptavidin-acceptor bead and Protein A-donor bead together.

FIG. 3 is a schematic illustration of an AlphaScreen assay. Streptavidin-acceptor bead and Protein A-donor bead are brought into proximity by the interaction between Ng66 and Fc-NgR to form a complex that emits light between 520 nm and 620 nm upon laser irradiation on the Protein A-donor bead at 680 nm.

FIG. 4 is a schematic illustration of an AlphaScreen assay. The interaction between Ng66 and Fc-NgR is blocked by a Nogo peptide fragment NEP33, preventing Streptavidin-acceptor bead and Protein A-donor bead from beingbrought into proximity to form a complex that emits light upon laser irradiation.

FIG. 5 is a graph depicting the AlphaScreen signals. The graph presents the effect of Ng66 and Fc-NgR concentrations on the intensity of AlphaScreen signals. Streptavidin-acceptor beads and Protein A-donor beads were incubated with Ng66 and Fc-NgR for 6 hours. The beads concentration is 6 μg/ml.

FIG. 6 is a graph depicting the AlphaScreen signals in the presence of a Nogo peptide fragment NEP1-33. The graph presents the effect of incubation time on the intensity of AlphaScreen signals. The beads concentration is 5 μg/ml. After 20 hours of incubation, the interaction of Ng66 and Fc-NgR is inhibited by NEP33 at a concentration between 1.25 μM and 20 μM.

FIG. 7 is a flow chart illustrating the steps to identify a small molecule compound that modulates the interaction of Nogo and Nogo receptor (NgR). By using an AiphaScreen, small molecule compounds (10 μM) are added to the wells of a 384-well microplate. The microplate contains the mixture of donor beads, acceptor beads, Fc-NgR and biotinylated Ng66. Compounds that inhibit the AiphaScreen signal are deemed hits and subsequently subjected to a “true hits” assay to detect their intrinsic signal quenching activities. Compounds that are detected with intrinsic signal quenching activity in the “true hits” assay are false positives. Compounds that pass the “true hits” assay are then subjected to a dose-response assay and functional assay to characterize their potency and activity.

FIG. 8 depicts the results of an AlphaScreen of Fc-NgR:Ng66 interaction. The graph presents 5 compounds that exhibit signal inhibition (hits) in the AlphaScreen of Fc-NgR:Ng66 interaction.

FIG. 9 depicts the results of AlphaScreen TruHits assay of compounds 10 and 12. Compounds 10 and 12 are members of a 20,000 small molecule library obtained from Maybridge Ltd. Both compounds exhibit hits in the AlphaScreen of Fc-NgR:Ng66 interaction. To validate the observed hits, a dilution series ranging from 10 uM to 0.000508 μM (final concentration) of each compound was added to aqueous wells containing AlphaScreen TruHits kit components. Dose dependent signal inhibition was observed in the AlphaScreen TruHits assay, indicating that compounds 10 and 12 are likely false positives.

FIG. 10 depicts secondary assay methods and results. FIG. 10A illustrate an ELISA assay; FIG. 10B illustrates a DELFIA assay; FIG. 10C presents the results of ELISA assay for NEP33 and Cisplatin; and FIG. 10D presents the results of DELFIA assay for NEP33. The dose-dependent inhibition of the NgR:Nogo ligand (Ng66) interaction-dependent signal in the presence of NEP33 and Cisplatin at the specified concentrations was determined.

FIG. 11A shows compound 4′-(7-methoxy-4,5-dihydropyrrolo[1,2-a]quinoxalin-4-yl)-N,N-dimethylaniline (“HTS”), and its dose dependent signal inhibition in the DELFIA assay of Fc-NgR:Ng66 interaction.

FIG. 11B shows compound 2-(4-chlorobenzoyl)-3-[4-(2-phenyleth-1-ynyl)phenyl]acrylonitrile (“KM”), and its dose dependent signal inhibition in the DELFIA of Fc-NgR:Ng66 interaction.

FIG. 11C shows compound 4-[(4-oxo-2-thioxo-1,3-thiazolan-3-yl)methyl]benzonitrile (“S”), and its dose dependent signal inhibition in the DELFIA of Fc-NgR:Ng66 interaction.

FIG. 12A shows the effect of Nogo Receptor on neurite outgrowth on postnatal day 10 (P10) mouse dorsal root ganglia (DRG) neurons in the wild-type and Nogo Receptor knockout mice. Quantification of neurite outgrowth is expressed as average neurite length (in microns) per neuron.

FIG. 12B shows the effect of Fc-NgR (7.5 μM) on neurite outgrowth on chick dorsal root ganglia (DRG) neurons. Quantification of neurite outgrowth is expressed as average neurite length (in microns) per neuron.

FIG. 13 shows the neurite outgrowth promoting effect of compound 2-(4-chlorobenzoyl)-3-[4-(2-phenyleth-1-ynyl)phenyl]acrylonitrile (“KM”). The top picture shows basal neurite outgrowth on embryonic day 13 (E13) chick DRG neurons in the presence of KM at a concentration of 20 μM, and the bottom picture shows basal neurite outgrowth in the absence of KM.

FIG. 14A depicts the effect of Fc-NgR (7.5 μM) on blocking the neurite outgrowth promoting effect of compound 2-(4-chlorobenzoyl)-3-[4-(2-phenyleth-1-ynyl)phenyl]acrylonitrile (“KM”). KM at the specified concentrations was co-incubated with Fc-NgR in embryonic day 13 (E13) chick DRG neuron culture for 3.5 hours. Quantification of neurite outgrowth is expressed on the Y axis as average neurite length (in microns) per neuron.

FIG. 14B depicts the effect of Fc-NgR (7.5 μM) on blocking the neurite outgrowth promoting effect of compound 4′-(7-methoxy-4,5-dihydropyrrolo[1,2-a]quinoxalin-4-yl)-N,N-dimethylaniline (“HTS”). HTS was co-incubated with Fc-NgR in embryonic day 13 (E13) chick DRG neuron culture for 3.5 hours Quantification of neurite outgrowth is expressed as average neurite length (in microns) per neuron.

FIG. 15 shows the neurite outgrowth inhibiting effect of compound 4-[(4-oxo-2-thioxo-1,3-thiazolan-3-yl)methyl]benzonitrile (“S”). The top picture shows no basal neurite outgrowth on E14 chick DRG neurons in the presence of compound S at a concentration of 20 μM, and the bottom picture shows basal neurite outgrowth in the absence of compound S.

FIG. 16 is a graph depicting the inhibitory effect of compound S on postnatal day 15 (P15) mouse DRG neurite outgrowth in the wild-type and Nogo Receptor knockout mice. Quantification of neurite outgrowth is expressed as average neurite length (in microns) per neuron.

FIG. 17A shows compound ethyl 5-[4-(dimethylamino)phenyl]-7-methyl-3-oxo-2,3-dihydro-5H-[1,3]thiazolo[3,2-a]pyrimidine-6-carboxylate (“555”), and its neurite outgrowth promoting effect on E13 chick DRG neurons at a concentration of 20 μM.

FIG. 17B depicts the effect of Fc-NgR (7.5 μM) on blocking neurite outgrowth promoting effect of compound 555. Compound 555 was co-incubated with Fc-NgR in embryonic day 13 (E13) chick DRG neuron culture for 3.5 hours. Quantification of neurite outgrowth is expressed as average neurite length (in microns) per neuron.

FIG. 18A shows compound (4-chlorophenyl)(5-hydroxy-1-benzofuran-3-yl)methanone (“5470”), and its neurite outgrowth promoting effect on embryonic day 14 (E14) chick DRG neurons at a concentration of 20 μM.

FIG. 18B shows compound (4-chlorophenyl)(5-hydroxy-2-methyl-1-benzofuran-3-yl)methanone (“585”), and its neurite outgrowth promoting effect on embryonic day 14 (E14) chick DRG neurons at a concentration of 20 μM.

FIG. 18C shows the effect of dimethylsulfoxide (control) on neurite outgrowth on embryonic day 14 (E14) chick DRG neurons.

FIG. 19A shows the effect of compound 5470 on neurite outgrowth on embryonic day 14 (E14) chick DRG neurons at a concentration of 20 μM. Quantification of neurite outgrowth is expressed as average neurite length (in microns) per neuron.

FIG. 19B shows the effect of compound 585 on neurite outgrowth on embryonic day 14 (E14) chick DRG neurons at a concentration of 20 μM. Quantification of neurite outgrowth is expressed as average neurite length (in microns) per neuron.

FIG. 20 shows the promoting or inhibitory effect of 29 compounds and dimethylsulfoxide (control) on the neurite outgrowth on embryonic day 13 (E13) chick DRG neurons at a concentration of 20 μM. Quantification of neurite outgrowth is expressed as average neurite length (in microns) per neuron. The compounds are compounds S, KM, HTS, 555, 510 (Chembridge Inc. catalog number 5106731), 516 (Chembridge Inc. catalog number 5162090), 524 (Chembridge Inc. catalog number 5249032), 535 (Chembridge Inc. catalog number 5352829), 536 (Chembridge Inc. catalog number 5363829), 5470 (Chembridge Inc. catalog number 5470065), 5472 (Chembridge Inc. catalog number 5472739), 5475 (Chembridge Inc. catalog number 5475092), 5476 (Chembridge Inc. catalog number 5476362), 560 (Chembridge Inc. catalog number 5607016), 561 (Chembridge Inc. catalog number 5611936), 585 (Chembridge Inc. catalog number 5851694), 592 (Chembridge Inc. catalog number 7110), 594 (Chembridge Inc. catalog number 5948019), 597 (Chembridge Inc. catalog number 5976525), 605 (Chembridge Inc. catalog number 6054710), 636 (Chembridge Inc. catalog number 6367674), 664 (Chembridge Inc. catalog number 6641843), 678 (Chembridge Inc. catalog number 6789717), 687 (Chembridge Inc. catalog number 6874781), 794 (Chembridge Inc. catalog number 7949736), 798 (Chembridge Inc. catalog number 7986605), KM01804, BTB11222 and KMO2502.

FIG. 21 shows that five compounds that promote neurite outgrowth have no effect on embryonic day 8 (E8) chick DRG neurons, which do not express Nogo receptor. The five compounds are KM, HTS, 555, 585, and 5470.

### DETAILED DESCRIPTION OF THE INVENTION

### Definitions and General Techniques

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

Although methods and materials similar or equivalent to those described herein can be used in practice or testing of the present invention, suitable methods and materials are described below. The materials, methods and examples are illustrative only, and are not intended to be limiting. Other features and advantages of the invention will be apparent from the detailed description and from the claims.

Throughout this specification and claims, the word “comprise,” or variations such as “comprises” or “comprising,” will be understood to imply the inclusion of a stated integer or group of integers but not the exclusion of any other integer or group of integers.

In order to further define this invention, the following terms and definitions are herein provided.

It is to be noted that the term “a” or “an” entity, refers to one or more of that entity; for example, “a small molecule,” is understood to represent one or more small molecules. As such, the terms “a” (or “an”), “one or more,” and “at least one” can be used interchangeably herein.

As used herein, the term “consists of,” or variations such as “consist of or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “NogoR fusion protein” means a protein comprising a soluble Nogo receptor-1 moiety fused to a heterologous polypeptide.

As used herein, “Nogo receptor,” “NogoR,” “NogoR-1,” “NgR,” “NgR-1,” “NgR1” and “NGR1” each means Nogo receptor-1.

A “small molecule library” or “library” is a collection of different compounds having different chemical structures. A small molecule library is screenable, that is, the compound library members therein may be subject to screening assays. In preferred embodiments, the library members can have a molecular weight of no more than 500 daltons, preferably from about 100 to about 350 daltons, or from about 150 to about 350 daltons.

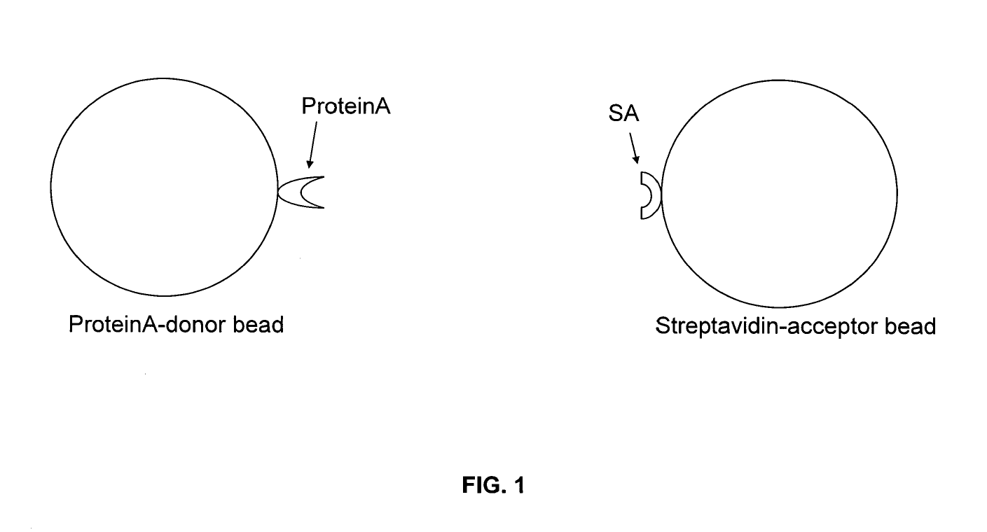

Libraries of candidate compounds can be assayed by many different assays, such as AlphaScreen assay (FIGS. 1-9). Libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized.

A “focused library” means that the collection of compounds is prepared using the structure of previously characterized compounds. The compounds in a “focused library” can be structurally similar or related to the previously characterized compounds. By “structurally similar or related” it is meant that the compounds share an attribute or a core structure.

A “small molecule library” useful for the invention may be purchased on the commercial market. The commercially suppliers include Chembridge Inc. or Maybridge Ltd. (a subsidiary of Maybridge Chemical Holdings Ltd.). A “small molecule library” used in the present invention was obtained from Maybridge Ltd. The library includes 20,000 compounds with a diverse struture.

A “small molecule library” useful for the invention can be prepared or obtained by any means including, but not limited to, combinatorial chemistry techniques, fermentation methods, plant and cellular extraction procedures and the like (e.g., Cwirla et al., Biochemistry 87: 6378-6382 (1990); Houghten et al., Nature 354: 84-86 (1991); Lam et al., Nature 354:82-84 (1991); Brenner et al., Proc. Natl. Acad. Sci. USA 89:5381-5383 (1992); Houghten R. A., Trends Genet. 9:235-239 (1993); Gallop et al., J. Med. Chem. 1994, 37:1233-1251 (1994); Gordon et al., J. Med. Chem. 1994, 37:1385-1401 (1994); Carell et al., Chem. Biol. 3:171-183 (1995); Lebl et al., Biopolymers 37:177-198 (1995)).

Libraries of a diverse molecules are prepared in order to obtain members having one or more pre-selected attributes that can be prepared by a variety of techniques, including but not limited to parallel array synthesis (Houghtor R. A., “Parallel array and mixture-based synthetic combinatorial chemistry: tools for the next millennium,” Annu. Rev. Pharmacol. Toxicol. 40:273-82(2000)); solution-phase combinatorial chemistry (Merritt, “Solution phase combinatorial chemistry,” Comb. Chem. High Throughput Screen 1998 1(2):57-72 (1998), and Sun, “Recent advances in liquid-phase combinatorial chemistry,” Comb. Chem. High Throughput Screen 1999 2(6):299-318 (1999)); synthesis on soluble polymer (Gravert et al., “Synthesis on soluble polymers: new reactions and the construction of small molecules,” Curr. Opin. Chem. Biol. 1997 1(1):107-13 (1997)).

Focused libraries can be designed with the help of sophisticated strategies involving computational chemistry (e.g., Kundu et al., “Combinatorial chemistry: polymer supported synthesis of peptide and non-peptide libraries,” Prog. Drug Res. 53:89-156 (1999)) and the use of structure-based ligands using database searching and docking, de novo drug design and estimation of ligand binding affinities (Joseph-McCarthy D., “Computational approaches to structure-based ligand design,” Pharmacol. & Ther. 84(2):179-91(1999); Kirkpatrick et al., “Structure-based drug design: combinatorial chemistry and molecular modeling,” Comb. Chem. High Throughput Screen. 2:211-21 (1999); Eliseev A. V. & Lehn J. M., “Dynamic combinatorial chemistry: evolutionary formation and screening of molecular libraries,” Curr. Top. Microbiol. & Immunol. 243:159-72 (1999)).

The term “pharmaceutically acceptable salt,” as used herein, refers to any salt (e.g., obtained by reaction with an acid or a base) of a compound of the present invention that is physiologically tolerated in the target animal (e.g., a mammal, such as a human). Salts of the compounds of the present invention may be derived from inorganic or organic acids and bases. Examples of suitable acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, boronic, malonic, sulfonic, picolinic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of suitable bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of suitable such salts include, but are not limited to: acetate, adipate, alginate, aspartate, benzoate, benzenesulfonate, bisulfate, borate, boronate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, nitrate, sulfate, picolinate, besylate, perchloriate, salicylate, phosphate, and the like. Other examples of suitable salts according to the invention include anions of the compounds of the present invention compounded with a suitable cation such as Na⁺, K⁺, Ca²⁺, Mg²⁺, Mn²⁺, NH₄⁺, and NW₄⁺ (wherein W is a C₁₋₄ alkyl group), and the like, including additional pharmaceutically acceptable salts that are well known in the art (see, e.g., Remington's Pharmaceutical Sciences, Mack Publishing Co., Easton, Pa., 19th ed. 1995) and others that are known to those of ordinary skill in the relevant arts. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “pharmaceutical composition” as used herein refers to a composition comprising one or more active pharmaceutical ingredients including, but not limited to, one or more compounds of the invention which can be used to treat, prevent or reduce the severity of a disease, disorder or condition in a subject, e.g., a mammal such as a human, that is suffering from, that is predisposed to, or that has been exposed to the disease, disorder or condition. A pharmaceutical composition generally comprises an effective amount of one or more active agents, e.g., a compound of the present invention, or a stereoisomer or mixture of stereoisomers thereof, and a pharmaceutically acceptable carrier. The pharmaceutical composition can also comprise a compound of the invention and one or more additional ingredients, including but not limited to one or more therapeutic agents (e.g., other Nogo receptor antagonists, e.g., other Nogo receptor agonists e.g., soluble Nogo receptor polypeptides).

The term “pharmaceutically acceptable carrier” encompasses any of the standard pharmaceutical carriers, buffers and excipients, including phosphate-buffered saline solution, water, and emulsions (such as an oil/water or water/oil: emulsion), and various types of wetting agents and/or adjuvants. Suitable pharmaceutical carriers and their formulations are described in Remington's Pharmaceutical Sciences, Mack Publishing Co., Easton, Pa., 19th ed. 1995. Preferred pharmaceutical carriers depend upon the intended mode of administration of the active agent. Typical modes of administration are described below.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “therapeutically effective amount,” as used herein, refers to an amount of a given therapeutic agent sufficient, at dosages and for periods of time necessary, to result in amelioration of one or more symptoms of a disorder or condition, or prevent appearance or advancement of a disorder or condition, or cause regression of or cure from the disorder or condition. A therapeutic result need not be a “cure”.

The term “therapeutic agent,” as used herein refers to any chemical substance that can be used in the treatment, management, prevention or amelioration of a disease, condition or disorder or one or more symptoms thereof. Suitable therapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices, and nucleotide sequences encoding biologically active proteins, polypeptides, or peptides), antibodies, synthetic or natural inorganic molecules, mimetic agents, and synthetic or natural organic molecules. In some embodiments, the therapeutic agent is one which is known to be useful for, or has been or is currently being used for, the treatment, management, prevention or amelioration of a condition or disorder or one or more symptoms thereof.

Compounds of the present invention include its pharmaceutically acceptable salt as described above. Compounds of the present invention exist as stereoisomers including optical isomers. The invention includes all stereoisomers, as pure individual stereoisomer preparations and as enriched preparations of each, and as the racemic mixtures of such stereoisomers as well as the individual enantiomers and diastereomers that may be separated according to methods that are well-known to those of skill in the art.

Compounds of the present invention exist as amorphous or crystalline form, the invention includes the amorphous and all the polymorphs of the compounds.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

The invention is directed to certain NgR1 antagonists that promote neuronal survival, neurite outgrowth and axonal regeneration of neurons, for example, CNS neurons. For example, the present invention provides NgR1 small molecules which stimulate axonal growth under conditions in which axonal growth is normally inhibited. Thus, NgR1 antagonists of the invention are useful in treating injuries, diseases or disorders that can be alleviated by promoting neuronal survival, or by the stimulation of axonal growth or regeneration.

Exemplary CNS diseases, disorders or injuries include, but are not limited to, multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Globoid cell Leucodystrophy (Krabbe's disease) and Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, epilepsy and Bell's palsy.

The invention is directed to certain NgR1 agonists that inhibit neuronal survival, neurite outgrowth and axonal regeneration of neurons, for example, CNS neurons and methods for treating a disease, disorder or injury associated with hyper or hypo activity of neurons, abnormal neuron sprouting and/or neurite outgrowth, e.g., schizophrenia in an animal suffering from such disease. For example, the present invention provides NgR1 small molecules which inhibit axonal growth under conditions in which axonal growth is normally observed. Thus, NgR1 agonists of the invention are useful in treating Schizophrenia or schizoaffective disorders.

In addition, diseases or disorders which may be treated or ameliorated by the methods of the present invention include diseases, disorders or injuries which relate to the hyper- or hypo-activity of neurons, abnormal neuron sprouting, and/or abnormal neurite outgrowth. Such disease include, but are not limited to, schizophrenia, bipolar disorder, obsessive-compulsive disorder (OCD), Attention Deficit Hyperactivity Disorder (ADHD), Downs Syndrome, and Alzheimer's disease.

### Nogo Receptor-1

In some embodiments, the present invention is directed to the use of small molecules for promoting neurite outgrowth, promoting neuronal survival, promoting axonal survival, or inhibiting signal transduction by the NgR1 signaling complex. In some embodiments, the present invention is directed to the use of small molecules to inhibit neuronal survival, neurite outgrowth and axonal regeneration of neurons.

The human NgR1 polypeptide is shown below as SEQ ID NO:1.

Full-Length Human NgR1 (SEQ ID NO: 1):
MKRASAGGSRLLAWVLWLQAWQVAAPCPGACVCYNEPKVTTSCPQQGL
QAVPVGIPAASQRIFLHGNRISHVPAASFRACRNLTILWLHSNVLARI
DAAAFTGLALLEQLDLSDNAQLRSVDPATFHGLGRLHTLHLDRCGLQE
LGPGLFRGLAALQYLYLQDNALQALPDDTFRDLGNLTHLFLHGNRISS
VPERAFRGLHSLDRLLLHQNRVAHVHPHAFRDLGRLMTLYLFANNLSA
LPTEALAPLRALQYLRLNDNPWVCDCRARPLWAWLQKFRGSSSEVPCS
LPQRLAGRDLKRLAANDLQGCAVATGPYHPIWTGRATDEEPLGLPKCC
QPDAADKASVLEPGRPASAGNALKGRVPPGDSPPGNGSGPRHINDSPF
GTLPGSAEPPLTAVRPEGSEPPGFPTSGPRRRPGCSRKNRTRSHCRLG
QAGSGGGGTGDSEGSGALPSLTCSLTPLGLALVLWTVLGPC

The rat NgR1 polypeptide is shown below as SEQ ID NO:2.

Full-Length Rat NgR1 (SEQ ID NO: 2):
MKRASSGGSRLLAWVLWLQAWRVATPCPGACVCYNEPKVTTSCPQQGL
QAVPTGIPASSQRIFLHGNRISHVPAASFQSCRNLTILWLHSNALARI
DAAAFTGLTLLEQLDLSDNAQLHVVDPTTFHGLGHLHTLHLDRCGLRE
LGPGLFRGLAALQYLYLQDNNLQALPDNTFRDLGNLTHLFLHGNRIPS
VPEHAFRGLHSLDRLLLHQNHVARVHPHAFRDLGRLMTLYLFANNLSM
LPAEVLMPLRSLQYLRLNDNPWVCDCRARPLWAWLQKFRGSSSEVPCN
LPQRLADRDLKRLAASDLEGCAVASGPFRPIQTSQLTDEELLSLPKCC
QPDAADKASVLEPGRPASAGNALKGRVPPGDTPPGNGSGPRHINDSPF
GTLPSSAEPPLTALRPGGSEPPGLPTTGPRRRPGCSRKNRTRSHCRLG
QAGSGASGTGDAEGSGALPALACSLAPLGLALVLWTVLGPC

The mouse NgR1 polypeptide is shown below as SEQ ID NO:3.

Full-Length Mouse NgR1 (SEQ ID NO: 3):
MKRASSGGSRLLAWVLWLQAWRVATPCPGACVCYNEPKVTTSCPQQGL
QAVPTGIPASSQRIFLHGNRISHVPAASFQSCRNLTILWLHSNALARI
DAAAFTGLTLLEQLDLSDNAQLHVVDPTTFHGLGHLHTLHLDRCGLRE
LGPGLFRGLAALQYLYLQDNNLQALPDNTFRDLGNLTHLFLHGNRIPS
VPEHAFRGLHSLDRLLLHQNHVARVHPHAFRDLGRLMTLYLFANNLSM
LPAEVLMPLRSLQYLRLNDNPWVCDCRARPLWAWLQKFRGSSSEVPCN
LPQRLADRDLKRLAASDLEGCAVASGPFRPIQTSQLTDEELLSLPKCC
QPDAADKASVLEPGRPASAGNALKGRVPPGDTPPGNGSGPRHINDSPF
GTLPSSAEPPLTALRPGGSEPPGLPTTGPRRRPGCSRKNRTRSHCRLG
QAGSGASGTGDAEGSGALPALACSLAPLGLALVLWTVLGPC

Full-length Nogo receptor-1 consists of a signal sequence, a N-terminus region (NT), eight leucine rich repeats (LRR), a LRRCT region (a leucine rich repeat domain C-terminal of the eight leucine rich repeats), a C-terminus region (CT) and a GPI anchor (see FIG. 1).

The NgR domain designations used herein are defined as follows:

TABLE 1
Example NgR domains

| | hNgR (SEQ | rNgR (SEQ | mNgR (SEQ |
|---|---|---|---|
| Domain | ID: 1) | ID NO: 2) | ID NO: 3) |
| Signal Seq. | 1-26 | 1-26 | 1-26 |
| LRRNT | 27-56 | 27-56 | 27-56 |
| LRR1 | 57-81 | 57-81 | 57-81 |
| LRR2 | 82-105 | 82-105 | 82-105 |
| LRR3 | 106-130 | 106-130 | 106-130 |
| LRR4 | 131-154 | 131-154 | 131-154 |
| LRR5 | 155-178 | 155-178 | 155-178 |
| LRR6 | 179-202 | 179-202 | 179-202 |
| LRR7 | 203-226 | 203-226 | 203-226 |
| LRR8 | 227-250 | 227-250 | 227-250 |
| LRRCT | 260-309 | 260-309 | 260-309 |
| CTS (CT | 310-445 | 310-445 | 310-445 |
| Signaling) | | | |
| GPI | 446-473 | 446-473 | 446-473 |

### Fusion Proteins and Conjugated Polypeptides

Some embodiments of the invention involve the use of an NgR1 polypeptide that is not the full-length NgR1 protein, e.g., polypeptide fragments of NgR1, fused to a heterologous polypeptide moiety to form a fusion protein. Such fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the NgR1 polypeptide moiety of the invention or to be cleavable, in vitro or in vivo. Heterologous moieties to accomplish these other objectives are known in the art.

As an alternative to expression of a fusion protein, a chosen heterologous moiety can be preformed and chemically conjugated to the NgR polypeptide moiety of the invention. In most cases, a chosen heterologous moiety will function similarly, whether fused or conjugated to the NgR polypeptide moiety. Therefore, in the following discussion of heterologous amino acid sequences, unless otherwise noted, it is to be understood that the heterologous sequence can be joined to the NgR polypeptide moiety in the form of a fusion protein or as a chemical conjugate.

Some embodiments of the invention employ an NgR polypeptide moiety fused to a hinge and Fc region, i.e., the C-terminal portion of an Ig heavy chain constant region. In some embodiments, amino acids in the hinge region may be substituted with different amino acids. Exemplary amino acid substitutions for the hinge region according to these embodiments include substitutions of individual cysteine residues in the hinge region with different amino acids. Any different amino acid may be substituted for a cysteine in the hinge region. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, serine and alanine. Making such substitutions through engineering of a polynucleotide encoding the polypeptide fragment is well within the routine expertise of one of ordinary skill in the art.

Potential advantages of an NgR-polypeptide-Fc fusion include solubility, in vivo stability, and multivalency, e.g., dimerization. The Fc region used can be an IgA, IgD, or IgG Fc region (hinge-CH2—CH3). Alternatively, it can be an IgE or IgM Fc region (hinge-CH2—CH3—CH4). An IgG Fc region is generally used, e.g., an IgG1 Fc region or IgG4 Fc region. Materials and methods for constructing and expressing DNA encoding Fc fusions are known in the art and can be applied to obtain fusions without undue experimentation. Some embodiments of the invention employ a fusion protein such as those described in Capon et al., U.S. Pat. Nos. 5,428,130 and 5,565,335.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47 (1990). In some embodiments, the Fc region of one of IgA, IgD, IgE, or IgM, is used.

Identification of Compounds that Modulate the Interaction of Nogo and Nogo Receptor

This invention also provides a method of identifying compounds which modulate the interaction of Nogo and Nogo receptor (NgR). The methods include: (a) mixing a Nogo polypeptide, a NgR polypeptide and a test compound; (b) measuring an interference of the binding of said Nogo polypeptide to said NgR polypeptide in the presence of said compound, as compared to the binding of said Nogo polypeptide to said NgR polypeptide in the absence of said compound.

In some embodiments, such method is conducted by using an AlphaScreen. AlphaScreen is generally described in Seethala and Prabhavathi, “Homogeneous Assays: AlphaScreen, Handbook of Drug Screening,” Marcel Dekkar Pub. 2001, pp. 106-110. The present invention uses an AlphaScreen, where small molecule compounds (20 μM) are added to the wells of a 384-well microplate. The microplate contains the mixture of donor beads, acceptor beads, Fc-NgR and biotinylated Ng66. Compounds that inhibit the AlphaScreen signal are deemed hits. (FIGS. 1-8).

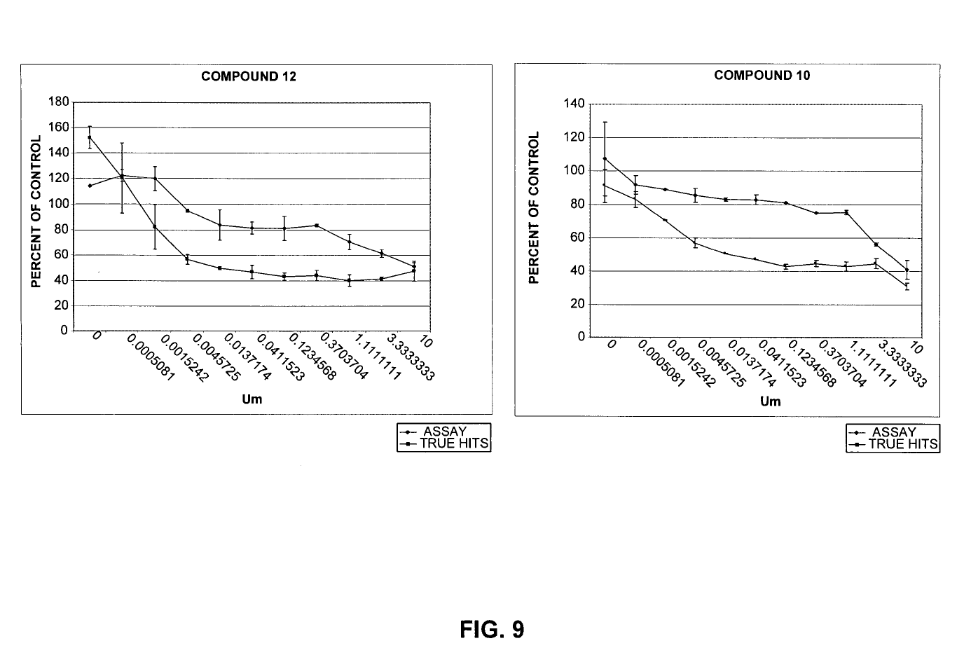

In some embodiments, the methods further include confirming the hits as described above by detecting an intrinsic signal interference of the compounds in the AlphaScreen by a dose-response assay, or a “true hits” assay. In some embodiments, the dose-response assay is performed by using AlphaScreen TruHits kit (PerkinElmer) to identify false positives in the AlphaScreen assay. The AlphaScreen TruHits kit allows the identification of classes of compounds including color quenchers, light scatterers (insoluble compounds), singlet oxygen quenchers and biotin mimetics that interfere with the AlphaScreen signal. Compounds which interfere with the AlphaScreen signal are considered false positives while compounds which exhibit no effect on the signal are potential true hits. (FIG. 9).

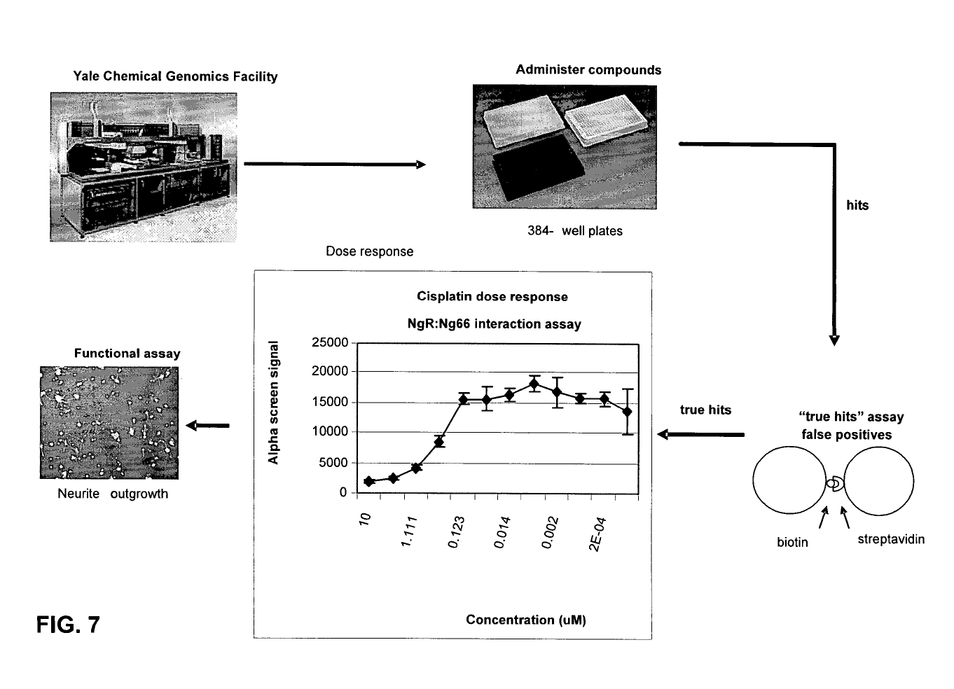

In some embodiments, the methods further include conducting a secondary dose-response assay and functional assay to characterize the potencies and activities of compounds that pass the “true hits” assay. (FIG. 7).

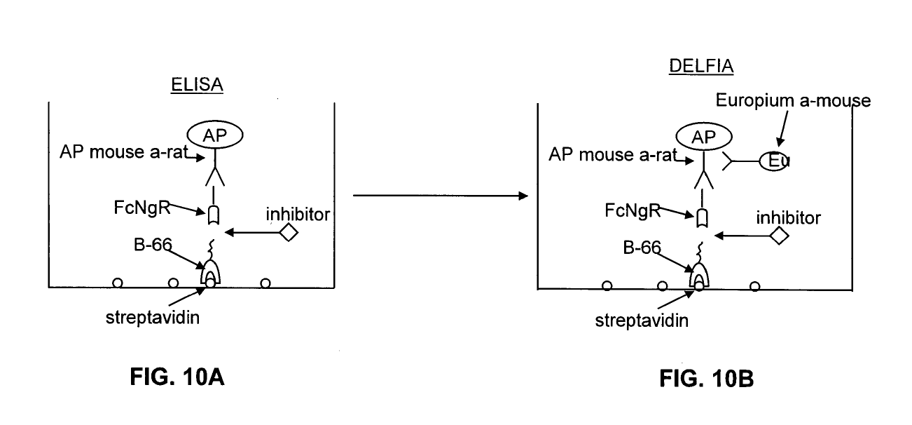

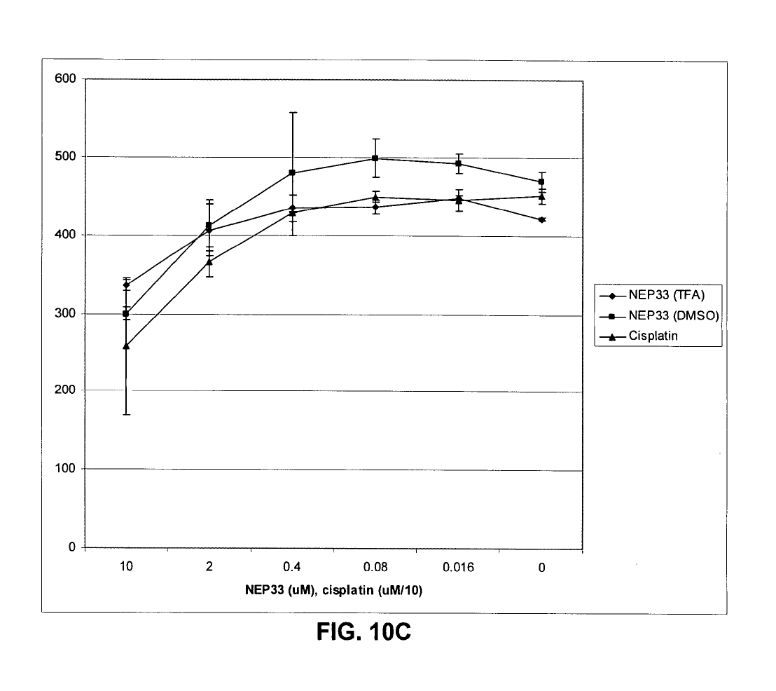

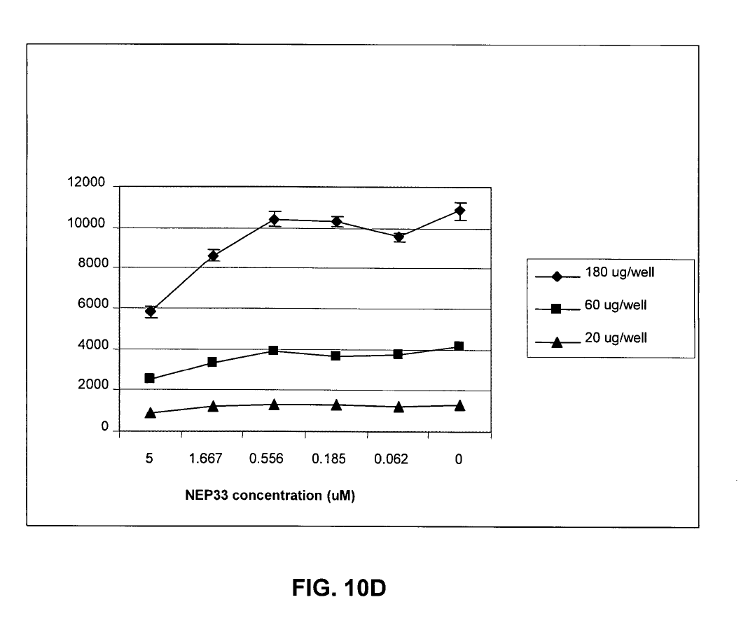

In some embodiments, the secondary dose-response assay is conducted by using Enzyme-Linked ImmunoSorbent Assay (ELISA) or Dissociation-Enhanced Lanthanide Fluorescent Immunoassay (DELFIA) to evaluate the ability of the compounds that are identified as “hits” in the AlphaScreens to inhibit the interaction of NgR and Nogo ligand. (FIGS. 10A-D and 11A-C).

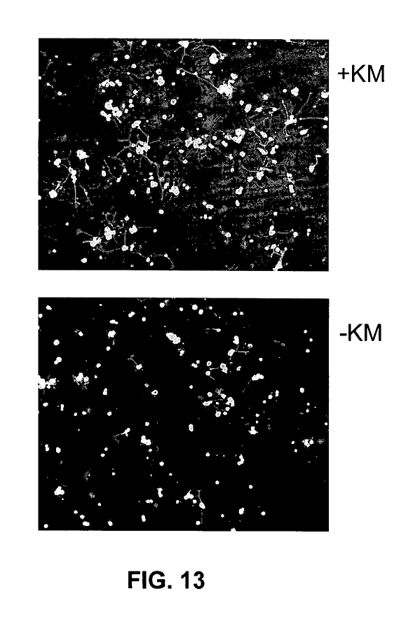

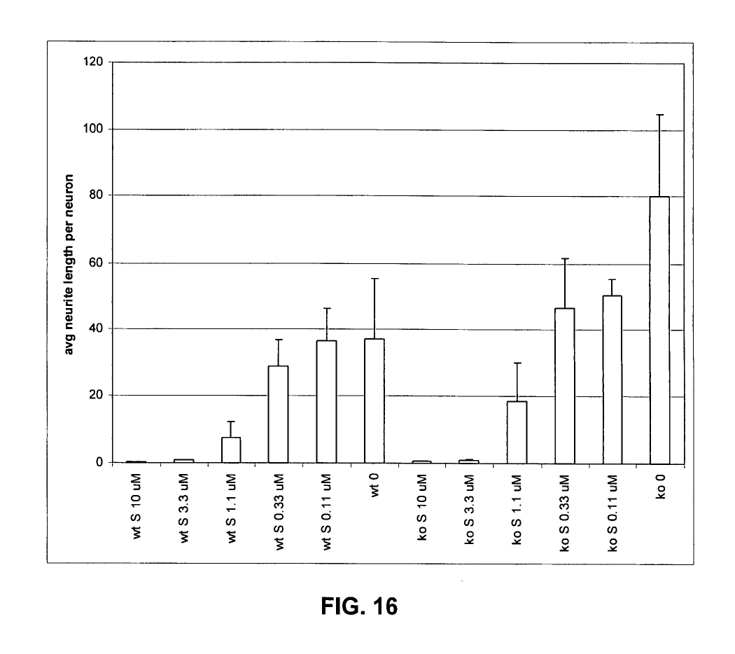

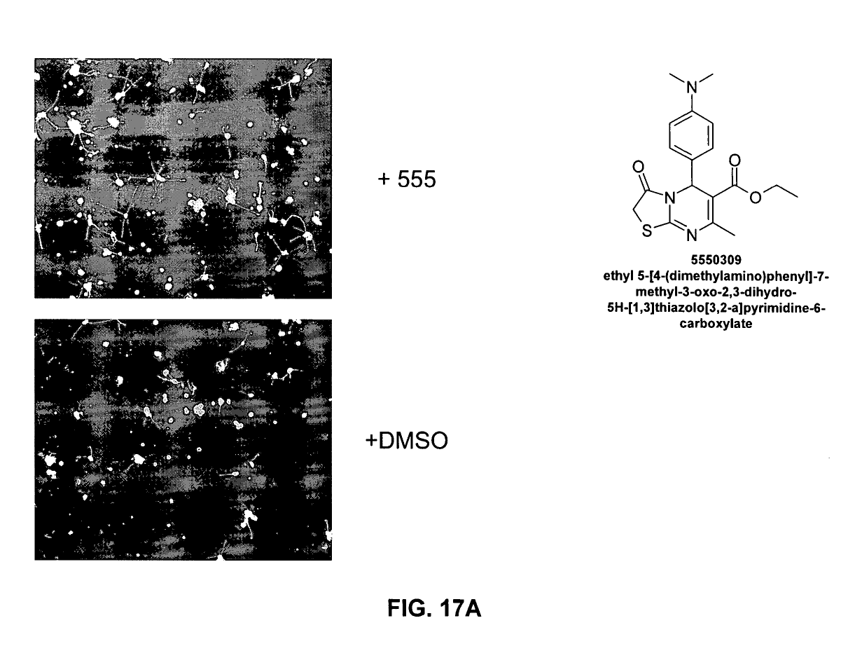

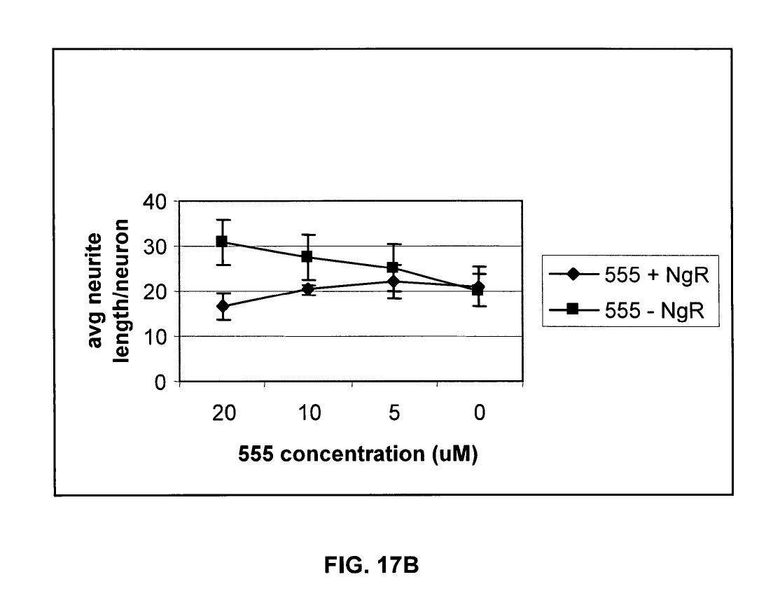

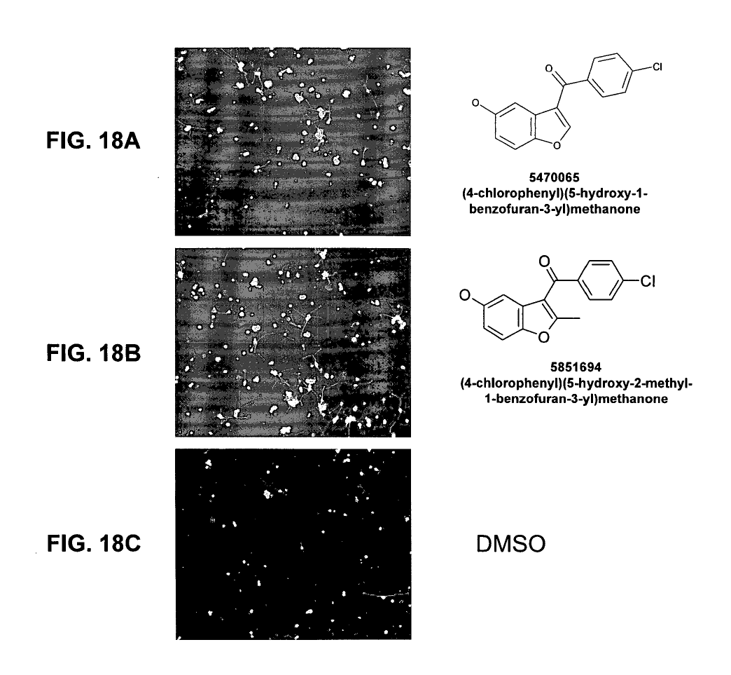

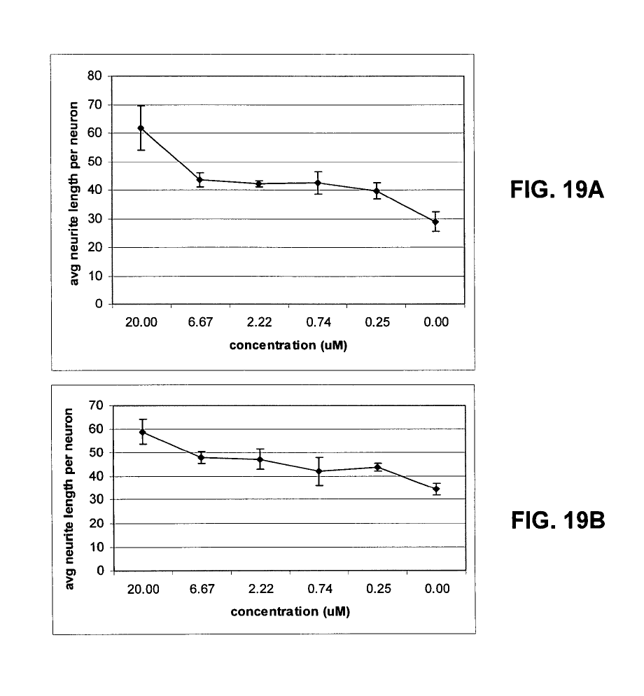

In some embodiments, the methods further include testing the ability of the “hit” compounds to promote or inhibit neurite outgrowth. (FIGS. 13-20).

Compounds that Modulate the Interaction of Nogo and Nogo Receptor

The present invention is directed to compounds that modulate the interaction of Nogo and Nogo receptor (NgR). The compounds of the invention include an optionally substituted, optionally partially saturated benzofuran, indole, thiazolopyrimidine, pyrroloquinoxaline, benzothiazole, chromene or quinoline.

The term “optionally substituted” as used herein means either unsubstituted or substituted with one or more substituents independently selected from hydroxy (OH), nitro (NO₂), cyano (CN), halo (F, Cl, Br, I), amino, alkyl, optionally substituted alkyl, optionally substituted cycloalkyl, optionally substituted alkenyl, optionally substituted alkynyl, optionally substituted heterocyclo, optionally substituted aryl, optionally substituted heteroaryl, alkoxy, aryloxy, aralkyloxy, acyl, aroyl, optionally substituted aroyl, optionally substituted aroylalkyl or alkoxycarbonyl.

The term “amino” as used herein refers to a radical of formula —NR^{a}R^{b} wherein R^{a} and R^{b} are independently hydrogen, optionally substituted alkyl, optionally substituted cycloalkyl, optionally substituted heterocyclo, optionally substituted aryl, optionally substituted heteroaryl or aralkyl; or R^{a} and R^{b} taken together with the nitrogen atom to which they are attached form a three to seven membered optionally substituted heterocyclo. Non-limiting exemplary amino groups include —NH₂, —N(H)CH₃, —N(CH₃)₂, —N(H)CH₂CH₃, —N(CH₂CH₃)₂ and the like.

The term “alkyl”, as used herein by itself or part of another group refers to a straight-chain or branched saturated aliphatic hydrocarbon typically having from one to eighteen carbons or the number of carbons designated. In one such embodiment, the alkyl is a C₁-C₆ alkyl. Non-limiting exemplary alkyl groups include methyl, ethyl, n-propyl, isopropyl, n-butyl, sec-butyl, isobutyl, tert-butyl, 4,4-dimethylpentyl and the like.

The term “optionally substituted alkyl” as used herein refers to that the alkyl as defined above is either unsubstituted or substituted with one or more substituents independently selected from hydroxy, nitro, cyano, halo, amino, optionally substituted cycloalkyl, optionally substituted aryl, optionally substituted heteroaryl, optionally substituted heterocyclo, alkoxy, aryloxy, aralkyloxy. In certain such embodiments, the substituents are selected from hydroxy, i.e., a hydroxyalkyl, halo, i.e., a haloalkyl, or amino, i.e., an aminoalkyl. Exemplary optionally substituted alkyl groups include —CH₂OCH₃, —CH₂CH₂CN, hydroxymethyl, hydroxyethyl, trifluoromethyl, benzyl, 4-cyanobenzyl, phenylethyl (i.e., PhCH₂CH—), (4-cyanophenyl)ethyl, diphenylmethyl (i.e., Ph₂CH—) and the like. Other suitable optionally substituted alkyl groups will be familiar to those of ordinary skill in the relevant arts.

The term “cycloalkyl” as used herein by itself or part of another group refers to saturated and partially unsaturated (containing one or two double bonds) cyclic hydrocarbon groups containing one to three rings typically having from three to twelve carbon atoms (i.e., C₃-C₁₂ cycloalkyl) or the number of carbons designated. Non-limiting exemplary cycloalkyl groups include cyclopropyl, cyclobutyl, cyclopentyl, cyclohexyl, cycloheptyl, cyclooctyl, norbornyl, decalin, adamantyl and the like. Other suitable cycloalkyl groups will be familiar to those of ordinary skill in the relevant arts.

The term “optionally substituted cycloalkyl” as used herein refers to the cycloalkyl as defined above is either unsubstituted or substituted with one or more substituents independently selected from halo, nitro, cyano, hydroxy, amino, optionally substituted alkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy acyl, aroyl, optionally substituted aroyl, optionally substituted aroylalkyl or alkoxycarbonyl. The term “optionally substituted cycloalkyl” also means that the cycloalkyl as defined above may be fused to an optionally substituted aryl. Non-limiting exemplary optionally substituted cycloalkyl groups include:

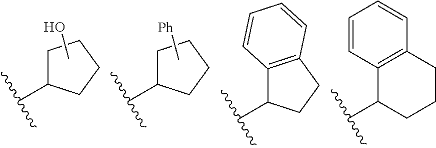

The term “alkenyl” as used herein by itself or part of another group refers to a group containing one or more carbon-to-carbon double bonds. Non-limiting exemplary alkenyl groups include —CH═CH— and the like. Other suitable alkenyl groups will be familiar to those of ordinary skill in the relevant arts.

The term “optionally substituted alkenyl” as used herein refers to the alkenyl as defined above is either unsubstituted or substituted with one or more substituents independently selected from halo, nitro, cyano, hydroxy, amino, optionally substituted alkyl, optionally substituted cycloalkyl, optionally substituted alkynyl, optionally substituted aryl, optionally substituted heteroaryl, optionally substituted heterocyclo, alkoxy, aryloxy, aralkyloxy acyl, aroyl, optionally substituted aroyl, optionally substituted aroylalkyl or alkoxycarbonyl. Non-limiting exemplary optionally substituted alkenyl groups include PhCH═CH— and the like. Other suitable optionally substituted alkenyl groups will be familiar to those of ordinary skill in the relevant arts.

The term “alkynyl” as used herein by itself or part of another group refers to group containing one more carbon-to-carbon triple bonds. Non-limiting exemplary alkynyl groups include —C≡C— and the like. Other suitable alkynyl groups will be familiar to those of ordinary skill in the relevant arts.

The term “optionally substituted alkynyl” as used herein by itself or part of another group means the alkynyl as defined above is either unsubstituted or substituted with one or more substituents independently selected from halo, nitro, cyano, hydroxy, amino, optionally substituted alkyl, optionally substituted cycloalkyl, optionally substituted alkenyl, optionally substituted aryl, optionally substituted heteroaryl, optionally substituted heterocyclo, alkoxy, aryloxy, aralkyloxy acyl, aroyl, optionally substituted aroyl, optionally substituted aroylalkyl or alkoxycarbonyl. Non-limiting exemplary optionally substituted alkenyl groups include PhC≡C— and the like. Other suitable optionally substituted alkynyl groups will be familiar to those of ordinary skill in the relevant arts.

The term “aryl” as used herein by itself or part of another group refers to monocyclic and bicyclic aromatic ring systems typically having from six to fourteen carbon atoms (i.e., C₆-C₁₄ aryl) such as phenyl (abbreviated as Ph), 1-naphthyl, and the like. Other aryl groups suitable for use in accordance with this aspect of the invention will be familiar to those of ordinary skill in the relevant arts.

The term “optionally substituted aryl” as used herein means the aryl as defined above is either unsubstituted or substituted with one or more substituents independently selected from halo, nitro, cyano, hydroxy, amino, optionally substituted alkyl, optionally substituted cycloalkyl, optionally substituted alkenyl, optionally substituted alkynyl, optionally substituted heteroaryl, optionally substituted heterocyclo, alkoxy, aryloxy, aralkyloxy acyl, aroyl, optionally substituted aroyl, optionally substituted aroylalkyl or alkoxycarbonyl. In one such embodiment, the optionally substituted aryl is an optionally substituted phenyl, which in certain embodiments has one or more substituents. Non-limiting exemplary substituted aryl groups include 4-dimethylaminophenyl, 4-diethylaminophenyl, 4-hydroxyphenyl, 4-cyanophenyl, 4-chlorophenyl, 4-methoxyphenyl, and the like. As used herein, the term “optionally substituted aryl” is also meant to include groups having fused optionally substituted cycloalkyl and fused optionally substituted heterocyclo rings. Non-limiting exemplary examples include:

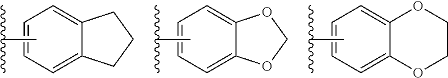

The term “aralkyl” as used herein by itself or part of another group refers to an optionally substituted alkyl as defined above having one or more optionally substituted aryl substituents. In one such embodiment, the optionally substituted alkyl is unsubstituted. In certain such embodiments, the optionally substituted aryl is phenyl (abbreviated as “Ph”). Non-limiting exemplary aralkyl groups include benzyl, 4-cyanobenzyl, phenylethyl, (4-cyanophenyl)ethyl, diphenylmethyl, (4-fluorophenyl)ethyl, and the like. Other suitable aralkyl groups will be familiar to those of ordinary skill in the relevant arts.

The term “heteroaryl” as used herein by itself or part of another group refers to monocyclic and bicyclic aromatic ring systems typically having from five to fourteen carbon atoms (i.e., C₅-C₁₄ heteroaryl) and one, two, three or four heteroatoms independently selected from the group consisting of oxygen, nitrogen and sulfur. In one such embodiment, the heteroaryl has four heteroatoms. In another such embodiment, the heteroaryl has three heteroatoms. In another such embodiment, the heteroaryl has two heteroatoms. In another such embodiment, the heteroaryl has one heteroatom. Non-limiting exemplary heteroaryl groups include 1-pyrrolyl, 2-pyrrolyl, 3-pyrrolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, purinyl, 2-benzimidazolyl, 4-benzimidazolyl, 5-benzimidazolyl, 2-benzthiazolyl, 4-benzthiazolyl, 5-benzthiazolyl, 5-indolyl, 3-indazolyl, 4-indazolyl, 5-indazolyl, 1-isoquinolyl, 5-isoquinolyl, 2-quinoxalinyl, 5-quinoxalinyl, 2-quinolyl, 3-quinolyl, 6-quinolyl, and the like. As used herein, the term “heteroaryl” is also meant to include possible N-oxides. Non-limiting exemplary N-oxides include pyridyl N-oxide and the like. Additional suitable heteroaryl groups for use in accordance with this aspect of the invention will be familiar to those of ordinary skill in the relevant arts.

The term “optionally substituted heteroaryl” as used herein means the heteroaryl as defined above is either unsubstituted or substituted with one or more substituents independently selected from halo, nitro, cyano, hydroxy, amino, optionally substituted alkyl, optionally substituted cycloalkyl, optionally substituted alkenyl, optionally substituted alkynyl, optionally substituted aryl, optionally substituted heterocyclo, alkoxy, aryloxy, aralkyloxy acyl, aroyl, optionally substituted aroyl, optionally substituted aroylalkyl or alkoxycarbonyl. Non-limiting exemplary optionally substituted heteroaryl groups include:

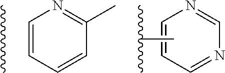

The term “heterocyclo” as used herein by itself or part of another group refers to saturated and partially unsaturated (containing one or two double bonds) cyclic groups containing one to three rings having from two to twelve carbon atoms (i.e., C₂-C₁₂ heterocyclo) and one or two oxygen, sulfur or nitrogen atoms. The heterocyclo can be optionally linked to the rest of the molecule through a carbon or nitrogen atom. Non-limiting exemplary heterocyclo groups include:

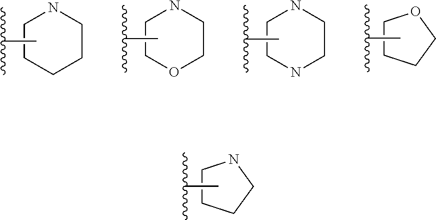

The term “optionally substituted heterocyclo” as used herein by itself or part of another group means the heterocyclo as defined above is either unsubstituted or substituted with one or more substituents independently selected from halo, nitro, cyano, hydroxy, amino, optionally substituted alkyl, optionally substituted cycloalkyl, optionally substituted alkenyl, optionally substituted alkynyl, optionally substituted aryl, optionally substituted heteroary, alkoxy, aryloxy, aralkyloxy acyl, aroyl, optionally substituted aroyl, optionally substituted aroylalkyl or alkoxycarbonyl. Substitution may occur on any available carbon or nitrogen atom.

The term “alkoxy” as used herein by itself or part of another group refers to an alkyl, optionally substituted alkyl, optionally substituted cycloalkyl, optionally substituted heterocyclo, optionally substituted alkenyl or optionally substituted alkynyl attached to a terminal oxygen atom. Non-limiting exemplary alkoxy groups include methoxy and the like.

The term “aryloxy” as used herein by itself or part of another group refers to an aryl, optionally substituted aryl or an optionally substituted heteroaryl attached to a terminal oxygen atom. Non-limiting exemplary aryloxy groups include phenoxy and the like.

The term “aralkyloxy” as used herein by itself or part of another group refers to an aralkyl attached to a terminal oxygen atom. Non-limiting exemplary aralkyloxy groups include benzyloxy and the like.

The term “acyl” as used here refers to a radical of formula RC(═O)—, wherein R is alkyl, optionally substituted alkyl, aralkyl, optionally substituted cycloalkyl, optionally substituted alkenyl or optionally substituted alkynyl. Non-limiting exemplary acyl groups include acetyl and the like.

The term “aroyl” as used here refers to a radical of formula RC(═O)—, wherein R is aryl, optionally substituted aryl, or optionally substituted heteroaryl. Non-limiting exemplary aroyl groups include benzoyl, 4-chlorobenzoyl and the like.

The term “aroylalkyl” as used here refers to a radical of formula R^{a}C(═O)R^{b}—, wherein R^{a} is aryl, optionally substituted aryl, or optionally substituted heteroaryl, wherein R^{b} is alkyl or optionally substituted alkyl. Non-limiting exemplary substituted aroylalkyl groups include:

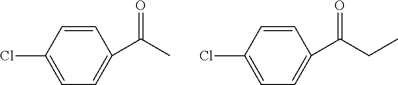

The term “alkoxycarbonyl” as used here refers to a radical of formula ROC(═O)—, wherein R is alkyl, optionally substituted alkyl, optionally substituted heterocyclo, aryl, optionally substituted aryl, or optionally substituted heteroaryl. Non-limiting exemplary alkoxycarbonyl groups include CH₃OC(═O)—, CH₃CH₂OC(═O)— and the like.

In some embodiments, an partially saturated indole, thiazolopyrimidine, pyrroloquinoxaline, benzothiazole, or chromene include:

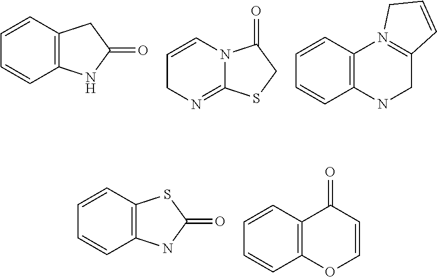

In some embodiments, the compounds that modulate the interaction of Nogo and Nogo receptor (NgR) include substituted benzofuran and quinoline. In some embodiments, the compounds that modulate the interaction of Nogo and Nogo receptor (NgR) include substituted partially saturated indole, thiazolopyrimidine, pyrroloquinoxaline, benzothiazole, or chromene as described above. The substitutions may occur on any available carbon or nitrogen atom. The exemplary substituents include benzoyl, 4-chlorobenzoyl, (4-chlorobenzoyl)ethyl, (4-cyanophenyl)ethyl, or 4-dimethylaminophenyl.

In some embodiments, the compounds that modulate the interaction of Nogo and Nogo receptor (NgR) include an optionally substituted 5-hydroxy-benzofuran, or an optionally substituted 5-hydroxy-3-aroylalkylbenzofuran. The substituents include halo, nitro, cyano, hydroxy, amino, optionally substituted alkyl, optionally substituted cycloalkyl, optionally substituted alkenyl, optionally substituted alkynyl, optionally substituted aryl, optionally substituted heteroary, alkoxy, aryloxy, aralkyloxy acyl, aroyl, optionally substituted aroyl, optionally substituted aroylalkyl or alkoxycarbonyl as described above. Substitutions may occur on any available carbon or nitrogen atom. The Non-limiting examples of such compounds in some embodiments include:

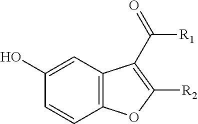

wherein R₁ is an optionally substituted aryl as defined above, and R₂ is hydrogen or an optionally substituted as defined above. In some embodiments, R₁ is 4-chlorophenyl. In some embodiments, R₂ is hydrogen or methyl. In some embodiments, such compounds have a molecular weight of no more than 500 daltons, and have no more than 5 nitrogen or 5 oxygen atoms.

In some embodiments, the compounds that modulate the interaction of Nogo and Nogo receptor (NgR) include an optionally substituted 3-acyl-indole, or an optionally substituted 3-hydroxy-3-aroylalkyl-1,3-dihydro-2H-indol-2-one. The substituents include halo, nitro, cyano, hydroxy, amino, optionally substituted alkyl, optionally substituted cycloalkyl, optionally substituted alkenyl, optionally substituted alkynyl, optionally substituted aryl, optionally substituted heteroary, alkoxy, aryloxy, aralkyloxy acyl, aroyl, optionally substituted aroyl, optionally substituted aroylalkyl or alkoxycarbonyl as described above. Substitutions may occur on any available carbon or nitrogen atom. Non-limiting examples of such compounds in some embodiments include:

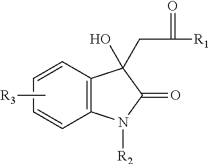

wherein R₁ is an optionally substituted aryl as defined above, R₂ is hydrogen or an optionally substituted alkyl, and R₃ is a halogen. In some embodiments, R₁ is 4-chlorophenyl. In some embodiments, R₂ is hydrogen, methyl or ethyl. In some embodiments, such compounds have a molecular weight of no more than 500 daltons, and have no more than 5 nitrogen or 5 oxygen atoms.
Compounds that Promote Neurite Outgrowth

The present invention is also directed to compounds that promote neurite outgrowth (FIGS. 13, 14A, 14B, 17A, 17B, 18A, 18B and 20). Such compounds are described in Table 2 and are available from Chembridge Inc. or Maybridge Ltd. (a subsidiary of Maybridge Chemical Holdings Ltd.).

TABLE 2
Compounds that Promote Neurite Outgrowth

| Compound Name | Compound Structure |
|---|---|
| 4′-(7-methoxy-4,5-dihydropyrrolo[1,2- a]quinoxalin-4-yl)-N,N-dimethylaniline (code name “HTS08871” or “HTS”) | 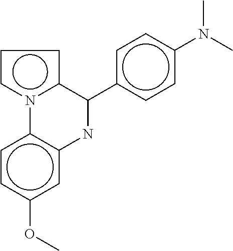 |
| 2-(4-chlorobenzoyl)-3-[4-(2-phenyleth-1- ynyl)phenyl]acrylonitrile (code name “KM08071” or “KM”) | 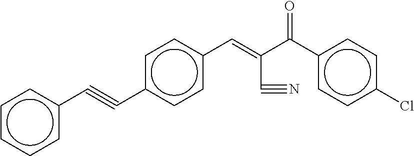 |
| ethyl 5-[4-(dimethylamino)phenyl]-7- methyl-3-oxo-2,3-dihydro-5H- [1,3]thiazolo[3,2-a]pyrimidine-6- carboxylate (Chembridge Inc. catalog number “5550309” or “555”) | 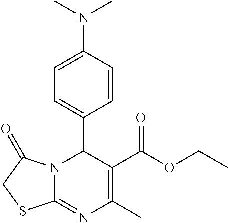 |
| (4-chlorophenyl)(5-hydroxy- 1-benzofuran- 3-yl)methanone (Chembridge Inc. catalog number “5470065” or “5470”) | 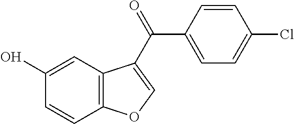 |
| (4-chlorophenyl)(5-hydroxy-2-methyl-1- benzofuran-3-yl)methanone (Chembridge Inc. catalog number “5851694” or “585”) | 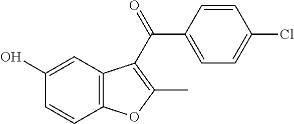 |
| 4-(1-benzoyl-1,2-dihydro-2-quinolinyl)- N,N-dimethylaniline (Chembridge Inc. catalog number: “5363829” or “536”) | 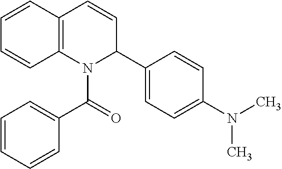 |
| 4-[(2-oxo-1,3-benzothiazol-3(2H)- yl)methyl]benzonitrile (Chembridge Inc. catalog number “5352829” or “535”) | 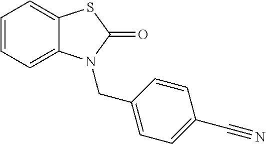 |
| 4-[(3-acetyl-7-ethyl-1H-indo1-1- yl)methyl]benzonitrile (Chembridge Inc. catalog number “7949736” or “794”) | 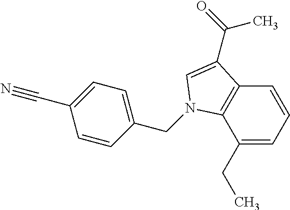 |
| 3-(4-chlorobenzoyl)-6-methyl-4H- chromen-4-one (Chembridge Inc. catalog number “5472749” or “5472”) | 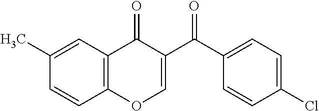 |
| N1,N1-dimethyl-4-[4- (dimethylamino)benzyl]aniline (code name “btb11222”) | 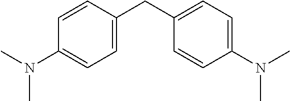 |

Compounds that Inhibit Neurite Outgrowth

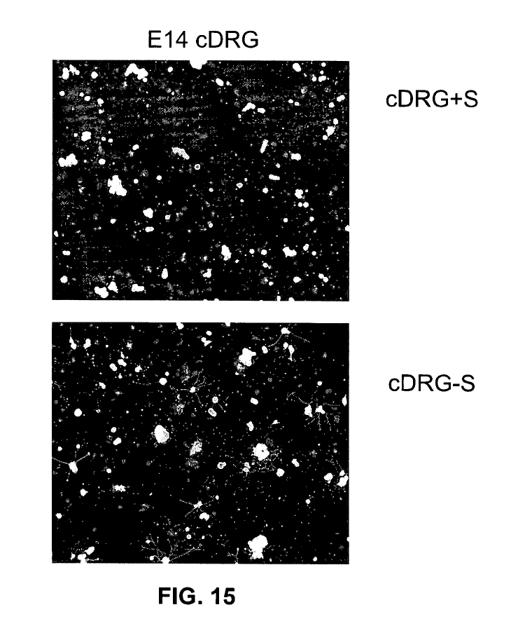

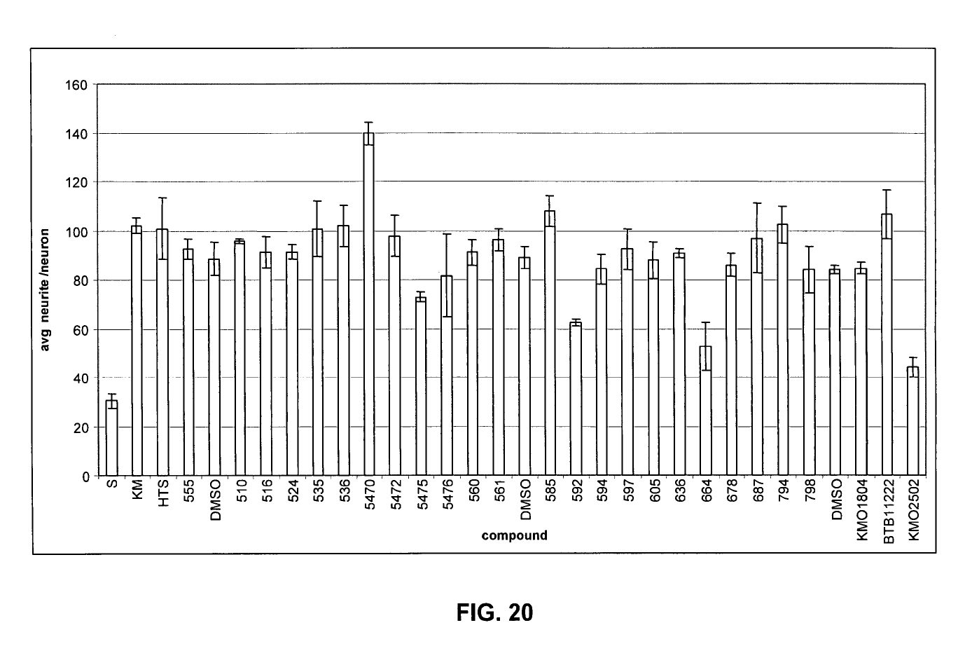

The invention is also directed to compounds that inhibit neurite outgrowth (FIGS. 15 and 20). Such compounds are described in Table 3 and are available from Chembridge Inc. or Maybridge Ltd. (a subsidiary of Maybridge Chemical Holdings Ltd.).

TABLE 3
Compounds that inhibit Neurite Outgrowth

| Compound Name | Compound Structure |
|---|---|
| 4-[(4-oxo-2-thioxo-1,3-thiazolan-3- yl)methyl]benzonitrile (code name “S03749”or “S”) | 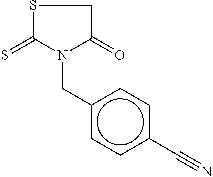 |
| 5-bromo-3-[2-(4-chlorophenyl)-2-oxoethyl]- 3-hydroxy-1,3-dihydro-2H-indol-2-one (Chembridge Inc. catalog number “5475092” or “5475”) | 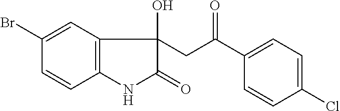 |
| 3-[2-(4-chlorophenyl)-2-oxoethyl]-1-ethyl-3- hydroxy-1,3-dihydro-2H-indol-2-one (Chembridge Inc. catalog number “6641843” or “664”) | 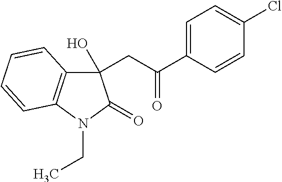 |
| 3-[2-(4-chlorophenyl)-2-oxoethyl]-3- hydroxy-1-methyl-1,3-dihydro-2H-indol-2- one (Chembridge Inc. catalog number “5927110” or “592”) | 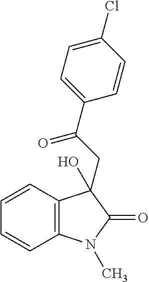 |
| 3-(4-chlorophenyl)-2-{2-[3-(2- methylprimidin-4-yl-phenyl]hydrazono}-3- oxopropanenitrile. (code name “KM02502”) | 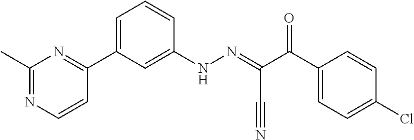 |

### Compositions

Compositions within the scope of the present invention include all compositions wherein one or more of the compounds of the present invention are contained in an amount which is effective to achieve its intended purpose. While individual needs vary, determination of optimal ranges of effective amounts of each component is within the expertise of those of ordinary skill in the art.

Compositions with the scope of the present invention also include all compositions wherein one or more of the compounds of the present invention are combined with one or more additional therapeutic agents (e.g., other Nogo receptor antagonists or agonists, e.g., soluble Nogo receptor polypeptides or anti-NgR antibodies) in therapeutically effective amounts. In addition to active agents (e.g., other Nogo receptor antagonists or agonists, e.g., soluble Nogo receptor polypeptides or anti-NgR1 antibodies), such compositions can optionally comprise one or more pharmaceutical excipients well-known in the relevant arts. The optimal amounts of each active agent in the composition can be determined by the clinical practitioner using routine methods known to the ordinarily skilled artisan based on the guidance provided herein and in view of the information that is readily available in the art.

In addition to administering the compound as a raw chemical, the compounds of the invention may be administered as part of a pharmaceutical composition comprising one or more compounds of the invention and one or more suitable pharmaceutically acceptable carriers, such as one or more excipients or auxiliaries which facilitate processing of the compounds into preparations which can be used pharmaceutically. Preferably, such pharmaceutical compositions contain from about 0.01 to 99 percent, e.g., from about 0.25 to 75 percent of active compound(s), together with the excipient(s), particularly those compositions which can be administered orally or topically and which can be used for the preferred type of administration, such as tablets, dragees, slow release lozenges and capsules, gels, liquid suspensions, as well as suitable solutions for administration by parenteral administration, e.g., via intravenous infusion, intramuscular, intracranial or subcutaneous injection.

The pharmaceutical compositions of the invention may be administered to any patient who may experience the beneficial effects of the compounds and/or compositions of the invention. Foremost among such patients are humans, although the invention is not intended to be so limited. Other patients include veterinary animals (cows, sheep, pigs, horses, dogs, cats and the like).

The compounds and pharmaceutical compositions of the invention may be administered by any means that achieve their intended purpose. For example, administration may be by parenteral, subcutaneous, intravenous, intramuscular, intradermal, intraperitoneal, transdermal, buccal, sublingual, intrathecal, intracerebroventricularly intracranial, intranasal, ocular, pulmonary (e.g., via inhalation), topical routes or direct infusion. Alternatively, or concurrently, administration may be by the oral route. The dosage administered will be dependent upon the age, health, and weight of the recipient, kind of concurrent treatment, if any, frequency of treatment, and the nature of the effect desired.

In the methods of the invention the compounds can be administered directly to the nervous system, intracerebroventricularly, or intrathecally, e.g. into a chronic lesion of MS. For treatment with a compound of the invention, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly.

In some methods, two or more therapeutic agents are administered simultaneously, in which case the dosage of each agent administered falls within the ranges indicated. Supplementary active compounds also can be incorporated into the compositions used in the methods of the invention. For example, a compound described herein may be coformulated with and/or coadministered with one or more additional therapeutic agents.

The invention encompasses any suitable delivery method for a compound to a selected target tissue, including bolus injection of an aqueous solution or implantation of a controlled-release system. Use of a controlled-release implant reduces the need for repeat injections.

The compounds used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-91 (1992); Gaspar et al., “Permanent 1251 Implants for Recurrent Malignant Gliomas,” Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

In some embodiments, a compound of the invention is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer a compound according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

The compositions may also comprise a compound of the invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

In certain embodiments, the compounds for use in the methods of the present invention further comprise a targeting moiety. Targeting moieties include a protein or a peptide which directs localization to a certain part of the body, for example, to the brain or compartments therein. In certain embodiments, compounds for use in the methods of the present invention are attached or fused to a brain targeting moiety. The brain targeting moieties are attached covalently (e.g., direct, translational fusion, or by chemical linkage either directly or through a spacer molecule, which can be optionally cleavable) or non-covalently attached (e.g., through reversible interactions such as avidin:biotin, protein A:IgG, etc.). In other embodiments, the compounds for use in the methods of the present invention thereof are attached to one more brain targeting moieties. In additional embodiments, the brain targeting moiety is attached to a plurality of compounds for use in the methods of the present invention.

A brain targeting moiety associated with a compound enhances brain delivery of such a compound. A number of polypeptides have been described which, when fused to a therapeutic agent, delivers the therapeutic agent through the blood brain barrier (BBB). Non-limiting examples include the single domain antibody FC5 (Abulrob et al. (2005) J. Neurochem. 95, 1201-1214); mAB 83-14, a monoclonal antibody to the human insulin receptor (Pardridge et al. (1995) Pharmacol. Res. 12, 807-816); the B2, B6 and B8 peptides binding to the human transferrin receptor (hTfR) (Xia et al. (2000) J. Virol. 74, 11359-11366); the OX26 monoclonal antibody to the transferrin receptor (Pardridge et al. (1991) J. Pharmacol. Exp. Ther. 259, 66-70); diptheria toxin conjugates. (see, for e.g., Gaillard et al., International Congress Series 1277:185-198 (2005); and SEQ ID NOs: 1-18 of U.S. Pat. No. 6,306,365. The contents of the above references are incorporated herein by reference in their entirety.

Enhanced brain delivery of a compound is determined by a number of means well established in the art. For example, administering to an animal a radioactively labelled compound linked to a brain targeting moiety; determining brain localization; and comparing localization with an equivalent radioactively labelled compound that is not associated with a brain targeting moiety. Other means of determining enhanced targeting are described in the above references.

Suitable oral pharmaceutical compositions of the present invention are manufactured in a manner which is itself well-known in the art, for example, by means of conventional mixing, granulating, dragee-making, dissolving, or lyophilizing processes. Thus, solid pharmaceutical preparations for oral use can be obtained by combining one or more of the compounds of the invention and optionally one or more additional active pharmaceutical ingredients with one or more solid excipients, optionally grinding the resulting mixture and processing the mixture of granules, after adding suitable auxiliaries, if desired or necessary, to obtain tablets or dragee cores.

Typically, the compounds may be administered to mammals, e.g., humans, orally at a dose of about 0.0025 to about 50 mg/kg, or an equivalent amount of the pharmaceutically acceptable salt, solvates or ester thereof. For example, about 0.01 to about 25 mg/kg can be orally administered to treat, ameliorate, or prevent such disorders. For intramuscular injection, the dose is generally about one-half of the oral dose, for example, a suitable intramuscular dose would be about 0.0025 to about 25 mg/kg, e.g., from about 0.01 to about 5 mg/kg.

The unit oral dose may comprise from about 0.01 to about 1000 mg of the compound or an equivalent amount of the pharmaceutically acceptable salt, solvates or ester thereof. The unit dose may be administered one or more times daily as one or more tablets or capsules.

In a topical formulation, the compound or its salts, solvates or esters may be present at a concentration of about 0.01 to 100 mg per gram of carrier.

Suitable excipients are, in particular, fillers such as saccharides, for example lactose, sucrose, fructose and the like; sugar alcohols such as mannitol, sorbitol, or xylitol and the like; cellulose preparations and/or calcium phosphates, for example tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or poly(ethylene glycol). Dragee cores are provided with suitable coatings which, if desired, are resistant to gastric juices. For this purpose, concentrated saccharide solutions may be used, which may optionally contain gum arabic, talc, polyvinyl pyrrolidone, poly(ethylene glycol) and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, can be used. Dye stuffs or pigments may be added to the tablets or dragee coatings, for example, for identification or in order to characterize combinations of active ingredients or doses thereof.

Other pharmaceutical preparations which can be used orally include push-fit capsules made of gelatin, as well as soft, sealed capsules made of gelatin and a plasticizer such as glycerol or sorbitol. In certain embodiments, the push-fit capsules can comprise one or more of the compounds of the invention in the form of granules which may be mixed with fillers such as lactose, binders such as starches, and/or lubricants such as talc or magnesium stearate and, optionally, stabilizers. In soft capsules, one or more pharmaceutical ingredients (e.g., one or more compounds of the invention and optionally one or more additional active pharmaceutical ingredients) are preferably dissolved or suspended in suitable liquids, such as fatty oils, or liquid paraffin. In addition, stabilizers may be added.

In addition to the solid dosage forms disclosed throughout, the present invention also provides chewable oral formulations. Such chewable formulations are especially useful in patient populations where compliance is an issue, such as children, the elderly, and patients who may have difficulty swallowing or using spray/inhalable formulations. In certain such embodiments, the formulations will comprise (or consist essentially of) an effective amount of one or more compounds of the invention along with suitable excipients that allow the formulations to be chewed by the patient. In additional embodiments, the formulations can further comprise one or more taste-masking or sweetening agents.

Any standard pharmaceutically acceptable excipient can be used in the chewable tablet formulations which provides adequate compression such as diluents (e.g., mannitol, xylitol, maltitol, lactitol, sorbitol, lactose, sucrose, and compressible sugars such as DiPac® (dextrinized sucrose), available from Austin Products Inc. (Holmdel, N.J.), binders, disintegrants, splitting or swelling agents (e.g., polyvinyl polypyrrolidone, croscarmellose sodium (e.g., Ac-Di-Sol available from FMC BioPolymer, Philadelphia, Pa.), starches and derivatives, cellulose and derivatives, microcrystalline celluloses, such as Avicel™ PH 101 or Avicel™ CE-15 (a microcrystalline modified with guar gum), both available from FMC BioPolymer, (Philadelphia, Pa.), lubricating agents (e.g., magnesium stearate), and flow agents (e.g., colloidal silicon dioxide, such as Cab-O-Sil M5® available from Cabot Corporation, Kokomo, Ind.).

In another embodiment, the present invention provides orally disintegrating/orodispersible tablets, such as those disclosed in U.S. Pat. No. 6,723,348, the disclosure of which is incorporated herein by reference in its entirety for all purposes. The orally disintegrating/orodispersible tablets suitably disintegrate in the buccal cavity upon contact with saliva forming an easy-to-swallow suspension. Such tablets comprise (or consist essentially of) compound(s) of the invention, and optionally, one or more additional active agents (such as those described herein), in the form of coated granules, and a mixture of excipients comprising at least one disintegrating agent, a soluble diluent agent, a lubricant and optionally a swelling agent, an antistatic (fluid flow) agent, a permeabilising agent, taste-masking agents/sweeteners, flavoring agents and colors. In certain such embodiments, the disintegrating/orodispersible tablets comprise the taste-masking agent sucralose. The amounts of compound(s) of the invention, other optional active agents, and sweetening agents (e.g., sucralose) in the orally disintegrating tablet formulations of the present invention are readily determinable by those of ordinary skill in the art, and include those amounts and combinations described herein.

In another embodiment, the present invention provides a solid, effervescent, rapidly dissolving dosage form of one or more compounds of the invention for oral administration, such as disclosed in U.S. Pat. No. 6,245,353, the disclosure of which is incorporated by reference herein in its entirety.

Another embodiment of the present invention is directed to a physiologically acceptable film that is particularly well-adapted to dissolve in the oral cavity of a warm-blooded animal including humans, and adhere to the mucosa of the oral cavity, to allow delivery of one or more compounds of the invention, and optionally one or more additional active agents such as those described herein. Such physiologically acceptable films suitable for use in accordance with this aspect of the present invention are disclosed in U.S. Patent Application No. 2004/0247648, the disclosure of which is incorporated herein by reference in its entirety.

Suitable formulations for oral and/or parenteral administration include aqueous solutions of one or more of the compounds of the invention, and optionally one or more additional active pharmaceutical ingredients, in water-soluble form, for example, water-soluble salts and alkaline solutions. In addition, suspensions of the active ingredient(s) as appropriate oily injection suspensions may be administered. Suitable lipophilic solvents or vehicles include fatty oils, for example, sesame oil, or synthetic fatty acid esters, for example, ethyl oleate or triglycerides or poly(ethylene glycol)-400. Aqueous injection suspensions may optionally also comprise substances which increase the viscosity of the suspension including, for example, sodium carboxymethyl cellulose, sorbitol, and/or dextran. Optionally, the suspension may also contain one or more stabilizers, one or more preservatives (e.g., sodium edetate, benzalkonium chloride, and the like), and/or other components commonly used in formulating pharmaceutical compositions.

Suitable topical pharmaceutical compositions of the invention are formulated preferably as oils, creams, lotions, ointments and the like by choice of appropriate carriers. Such compositions of the invention therefore comprise one or more compounds of the invention, optionally one or more additional active pharmaceutical ingredients, and one or more carriers suitable for use in preparing such pharmaceutical compositions for topical administration. Suitable such carriers include vegetable or mineral oils, white petrolatum (white soft paraffin), branched chain fats or oils, animal fats and high molecular weight alcohol (greater than C12). The preferred carriers are those in which the active pharmaceutical ingredient(s) are soluble. Emulsifiers, stabilizers, humectants and antioxidants may also be included, as well as agents imparting color or fragrance, if desired. Additionally, one or more transdermal penetration enhancers can be employed in these topical formulations. Non-limiting examples of suitable such enhancers can be found in U.S. Pat. Nos. 3,989,816 and 4,444,762, which are incorporated be reference herein in their relevant parts.

Suitable liquid pharmaceutical compositions for ocular administration comprise (or consisting essentially of) a therapeutically effective dose of one or more compounds of the invention, and one or more pharmaceutically acceptable carriers or excipients, wherein at least one of the pharmaceutically acceptable carriers or excipients is sucralose, wherein the composition is free, or substantially free of preservatives, and wherein the composition is provided in a single unit-dose container. Suitable unit-dose containers include, but are not limited to, high density polyethylene containers, for example, high density polyethylene containers produced using a blow-fill-seal manufacturing technique with a volume capacity of about 1 mL.

Suitable liquid pharmaceutical compositions for nasal administration in unit-dose or multi-dose configurations, comprising (or consisting essentially of) a therapeutically effective dose of one or more compounds of the invention, and one or more pharmaceutically acceptable carriers or excipients, wherein at least one of the pharmaceutically acceptable carriers or excipients is sucralose, wherein the composition is free, or substantially free of preservatives, and wherein the composition is provided in either a unit-dose or multi-dose container.

The present invention provides formulations and compositions for pulmonary delivery of one or more compounds of the invention, and optionally, one or more additional active agents, such as those described herein.

Suitable inhalable powder pharmaceutical compositions comprises (or consisting essentially of), a therapeutically effective dose of one or more compounds of the invention, and one or more pharmaceutically acceptable carriers or excipients, wherein the compound(s) of the invention are in the form of micronized particles and wherein at least one of the pharmaceutically acceptable carriers or excipients is sucralose, for example, micronized particles of sucralose. Suitable such inhalable powder pharmaceutical compositions comprise micronized particles of one or more compounds of the invention with an average particle size of about 1 μm to about 5 μm, and micronized particles of sucralose with an average particle size of about 1 μm to about 20 μm. Such inhalable powder pharmaceutical compositions of the present invention can be formulated for pulmonary delivery using, for example, a dry powder inhaler.

Suitable inhalable spray pharmaceutical compositions comprises (or consisting essentially of), a suitable concentration to provide a therapeutically effective dose of one or more compounds of the invention, and one or more pharmaceutically acceptable carrier, stabilizer or excipient, wherein the compound(s) of the invention is(are) in a solution form and wherein at least one of the pharmaceutically acceptable carriers or excipients is sucralose dissolved in the solution. Such inhalable spray pharmaceutical compositions when used with a suitable device provide a fine spray of the components (including active and non-active components) having an average particle size of about 1 μm to about 5 μm. Such inhalable spray pharmaceutical compositions of the present invention can be formulated for pulmonary delivery using, for example, a suitable device or inhaler.

In certain embodiments, a pharmaceutical composition comprising a compound of the invention and one or more additional therapeutic agents are administered to a patient.

In certain embodiments, compounds of the invention and one or more additional therapeutic agents are administered to a patient in separate compositions and are administered concurrently or at different periodicities.

In some embodiments, the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the molecules of this invention for delivery into the cell. Exemplary “pharmaceutically acceptable carriers” are any and all solvents, dispersion media, coatings, antibacterial and antifungal agents, isotonic and absorption delaying agents, and the like that are physiologically compatible, water, saline, phosphate buffered saline, dextrose, glycerol, ethanol and the like, as well as combinations thereof. In some embodiments, the composition comprises isotonic agents, for example, sugars, polyalcohols such as mannitol, sorbitol, or sodium chloride. In some embodiments, the compositions comprise pharmaceutically acceptable substances such as wetting or minor amounts of auxiliary substances such as wetting or emulsifying agents, preservatives or buffers, which enhance the shelf life or effectiveness of the compounds of the invention.

Compositions of the invention may be in a variety of forms, including, for example, liquid, semi-solid and solid dosage forms, such as liquid solutions (e.g., injectable and infusible solutions), dispersions or suspensions. The preferred form depends on the intended mode of administration and therapeutic application. In one embodiment, compositions are in the form of injectable or infusible solutions, such as compositions similar to those used for passive immunization of humans with other antibodies.

The composition can be formulated as a solution, micro emulsion, dispersion, liposome, or other ordered structure suitable to high drug concentration. Sterile injectable solutions can be prepared by incorporating a compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See e.g., Sustained and Controlled Release Drug Delivery Systems, J. R. Robinson, ed., Marcel Dekker, Inc., New York (1978).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage unit form as used herein refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound and the particular therapeutic or prophylactic effect to be achieved, and (b) the limitations inherent in the art of compounding such compound for the treatment of sensitivity in individuals. In some embodiments a therapeutically effective dose range for the compound of the invention is 0.0025-50 mg/Kg per day. In some embodiments a therapeutically effective dose range is 0.01-25 mg/Kg per day.

### Uses of the Compounds and Compositions

In some embodiments, the invention provides a method for promoting neurite outgrowth comprising contacting a neuron with a compound, or a composition of the invention. In some embodiments, the compound or composition inhibits neurite outgrowth inhibition. In some embodiments, the neuron is in a mammal. In some embodiments, the mammal is a human.

In some embodiments, the invention provides a method of inhibiting signal transduction by the NgR1 signaling complex, comprising contacting a neuron with an effective amount of a compound, or a composition of the invention. In some embodiments, the neuron is in a mammal. In some embodiments, the mammal is a human.

In some embodiments, the invention provides a method of treating a central neryous system (CNS) disease, disorder, or injury in a mammal, comprising administering to a mammal in need of treatment an effective amount of a compound or a composition of the present invention. In some embodiments, the disease, disorder, or injury is multiple sclerosis, ALS, Huntington's disease, Alzheimer's disease, Parkinson's disease, diabetic neuropathy, stroke, traumatic brain injuries, spinal cord injury, optic neuritis, glaucoma, hearing loss, and adrenal leukodystrophy.

In some embodiments, the invention provides a method for inhibiting neurite outgrowth comprising contacting a neuron with a compound, or a composition of the invention. In some embodiments, the compound or composition inhibits neurite outgrowth. In some embodiments, the neuron is in a mammal. In some embodiments, the mammal is a human.

In some embodiments, the invention provides a method of treating Schizophrenia or schizoaffective disorders in a mammal, comprising administering to a mammal in need of treatment an effective amount of a compound or a composition of the present invention.

Compounds of the present invention may be used therapeutically. In some embodiments, a compound of present invention is administered to a human patient. In some embodiments, a compound of present invention is administered to a non-human mammal expressing a Nogo receptor-1 for veterinary purposes or as an animal model of human disease. Such animal models may be useful for evaluating the therapeutic efficacy of compounds of this invention.

Compounds of the present invention can be provided alone, or in combination, or in sequential combination with other agents that modulate a particular pathological process. As used herein, the compounds of the present invention can be administered in combination with one or more additional therapeutic agents when the two are administered simultaneously, consecutively or independently.

Compounds of the present invention can be administered via parenteral, subcutaneous, intravenous, intramuscular, intraperitoneal, transdermal, inhalational or buccal routes. For example, an agent may be administered locally to a site of injury via microinfusion. Typical sites include, but are not limited to, damaged areas of the spinal cord resulting from injury. The dosage administered will be dependent upon the age, health, and weight of the recipient, kind of concurrent treatment, if any, frequency of treatment, and the nature of the effect desired.

Compounds of this invention can be utilized in vivo, ordinarily in mammals, such as humans, sheep, horses, cattle, pigs, dogs, cats, rats and mice, or in vitro.

It will be readily apparent to one of ordinary skill in the relevant arts that other suitable modifications and adaptations to the methods and applications described herein are obvious and may be made without departing from the scope of the invention or any embodiment thereof. In order that this invention may be better understood, the following examples are set forth. These examples are for purposes of illustration only and are not to be construed as limiting the scope of the invention in any manner.

### Examples

### Example 1

### Alpha Screen for Small Molecule Inhibitors of the Nogo Receptor: Nogo Ligand Interaction

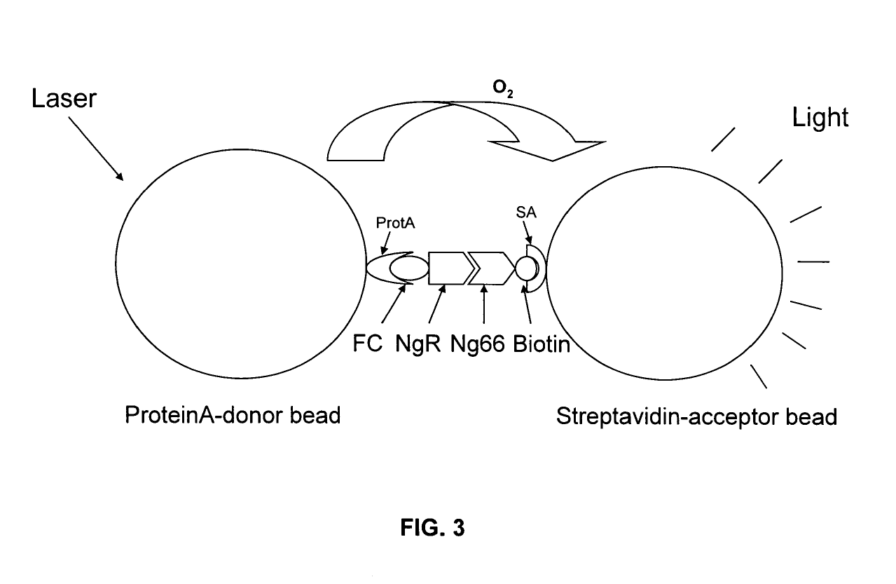

An AlphaScreen assay was used to screen for small molecule inhibitors of the Nogo receptor-Nogo ligand interaction. The AlphaScreen assay involves matching Alpha Donor (Streptavidin) and Acceptor beads (Protein A). (FIG. 1) These beads are coated with a layer of hydrogel to provide functional groups for bioconjugation. Streptavidin-acceptor beads and Protein A-donor beads in solution do not produce a signal by themselves. However, if a biological reaction brings the Alpha Donor and Acceptor beads into close proximity, upon laser excitation, a cascade of chemical reactions produces a greatly amplified signal. (FIG. 3) Upon laser excitation, a photosensitizer inside the Donor bead converts ambient oxygen to a more excited singlet state. The singlet state oxygen molecules diffuse to produce a chemiluminescent reaction in the Acceptor bead, leading to light emission. In the absence of a specific biological interaction, the singlet state oxygen molecules produced by the Donor bead go undetected without the close proximity of the Acceptor bead.

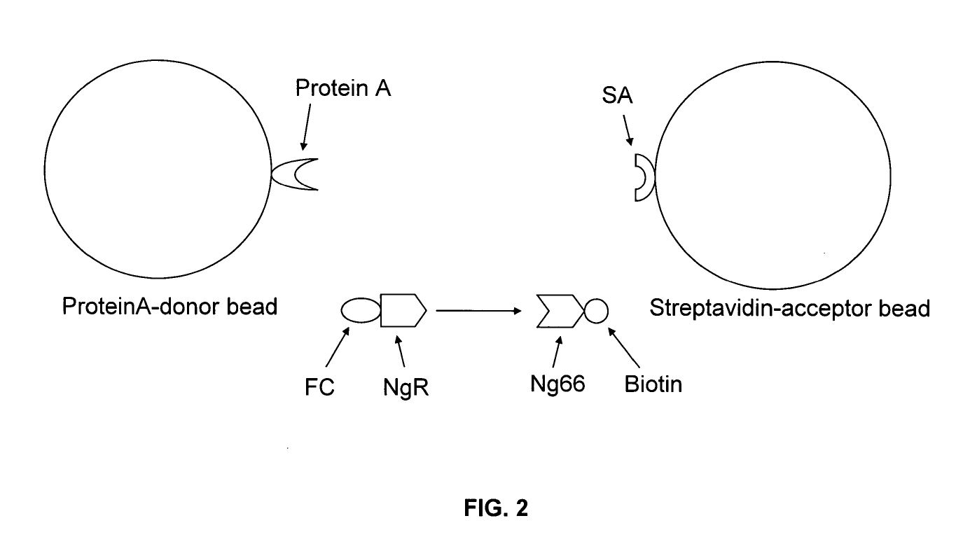

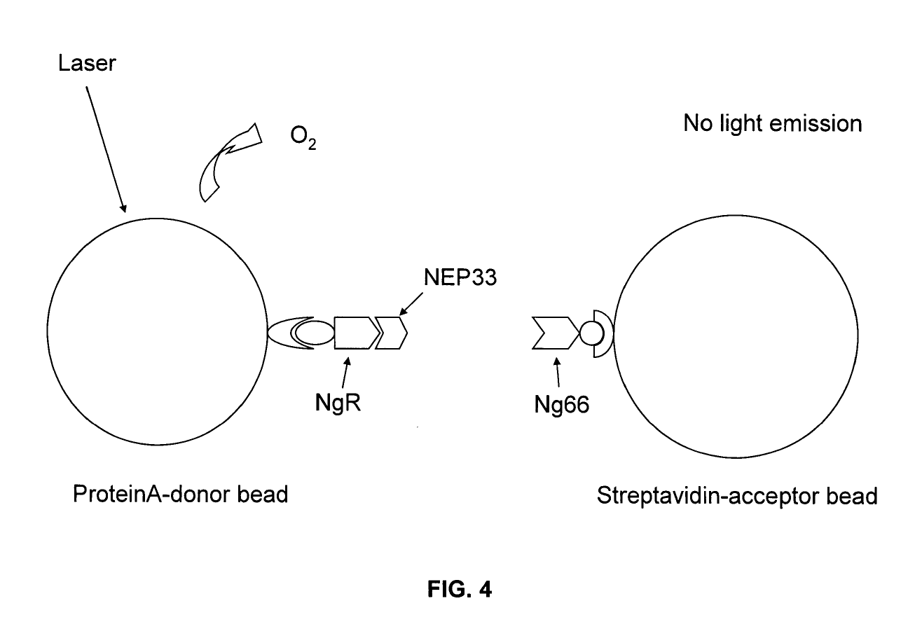

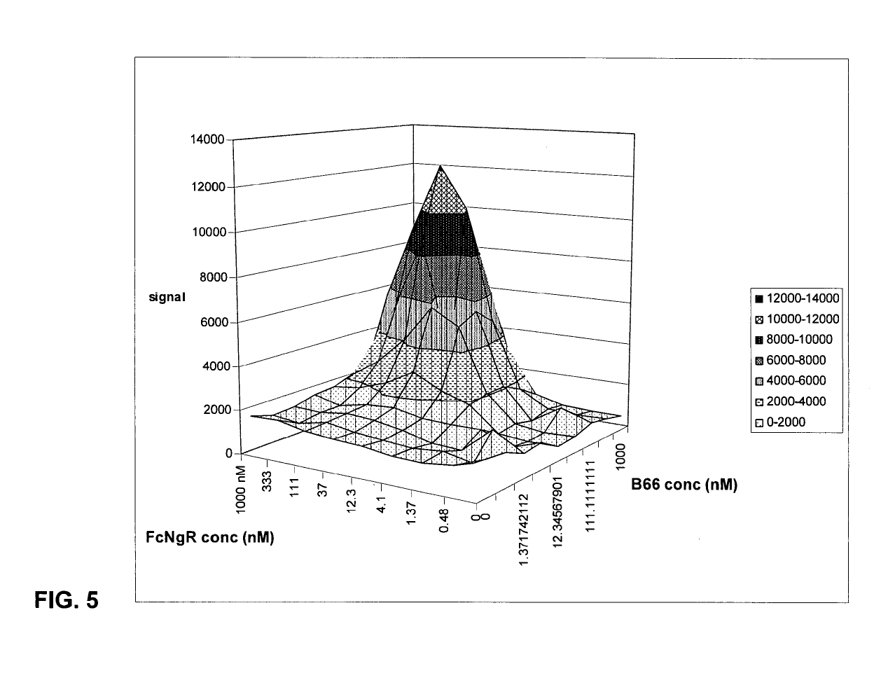

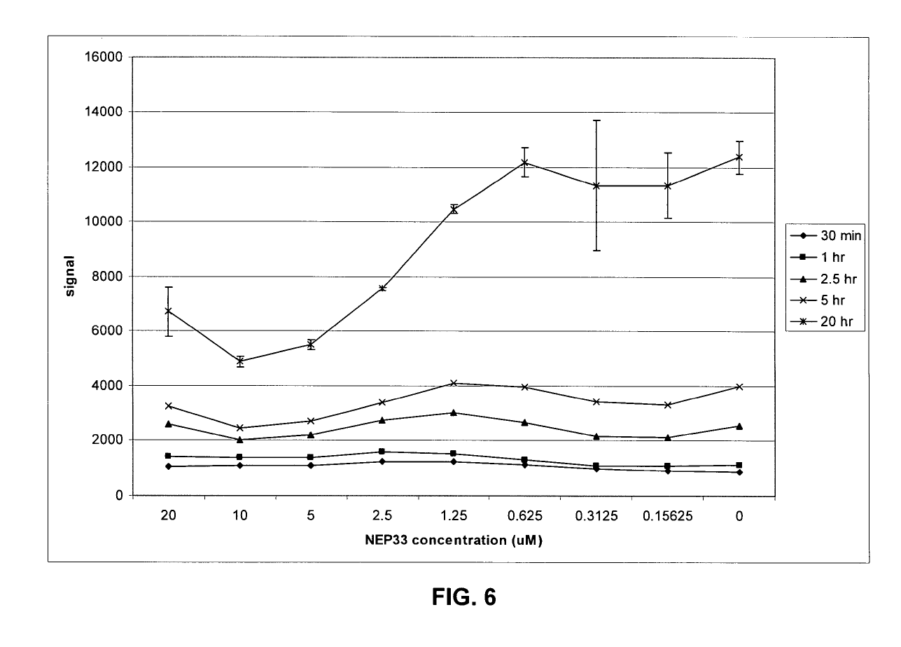

Beads conjugated to streptavidin were used to bind biotinylated Nogo 66 (Ng66) (the 66 amino acid inhibitory domain in the carboxyl region of Nogo ligand), and beads conjugated to Protein A were used to bind an Fc-Nogo receptor (NgR) fusion protein. (FIG. 2) By virtue of the interaction of Ng66 and Fc-NgR, the acceptor beads and donor beads were brought into proximity, yielding a signal upon excitation. (FIGS. 3 and 5) Molecules that interfered with the interaction, such as unbiotinylated Ng66 and NEP-33 (Acetyl-RIYKSVLQAVQKTDEGHPFKAYLELEITLSQEQ-Amide) (SEQ ID NO:4), prevented the beads from being brought into proximity, thus reducing signal as an indication of the interference. (FIGS. 4 and 6).

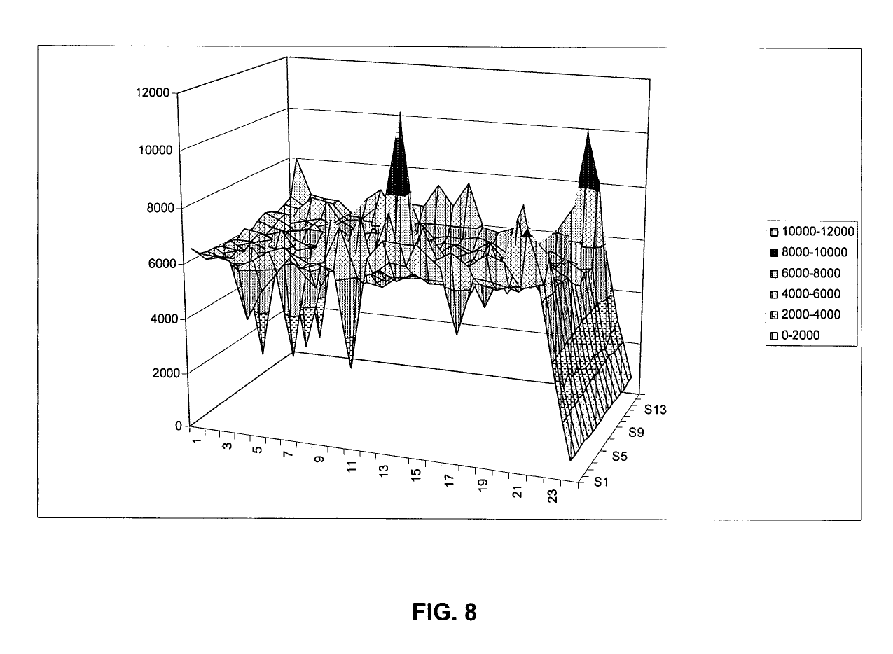

Twenty thousand (20,000) compounds were screened for reduction of the signal, and thus Fc-NgR:Ng66 interaction inhibiting activity. The compounds (10 uM) were added to wells containing the mix of donor beads, acceptor beads, Fc-NgR and biotinylated Ng66. An example plate showing 5 compounds that exhibited signal inhibition is shown in FIG. 8. Of the 20,000 compounds tested, 163 compounds had signal-inhibitory activity and were chosen for subsequent evaluation.

### Example 2

### Secondary TruHits Screen for Small Molecule Inhibitors of the Nogo Receptor:Nogo Ligand Interaction

AlphaScreen TruHits kit (PerkinElmer) was used to identify false positives in the AlphaScreen assay. The AlphaScreen TruHits kit allows the identification of classes of compounds including color quenchers, light scatterers (insoluble compounds), singlet oxygen quenchers and biotin mimetics that interfere with the AlphaScreen signal. Library compounds which interfere with the AlphaScreen signal are considered false positives while compounds which exhibit no effect on the signal are potential true hits.

Compounds 10 and 12 were identified as hits using the AlphaScreen. To evaluate their validity, these compounds were evaluated using the AlphaScreen TruHits kit according to manufacturer's instructions. A dilution series ranging from 10 uM to 0.000508 uM (final concentration) of each compound was added to aqueous wells containing AlphaScreen TruHits kit components. Dose dependent signal inhibition was observed in the AlphaScreen TruHits assay, indicating that compounds 10 and 12 are likely false positives. (FIG. 9).

### Example 3

### ELISA and DELFIA Assays to Evaluate Potential Small Molecule Inhibitors of the Nogo Receptor:Nogo Ligand Interaction

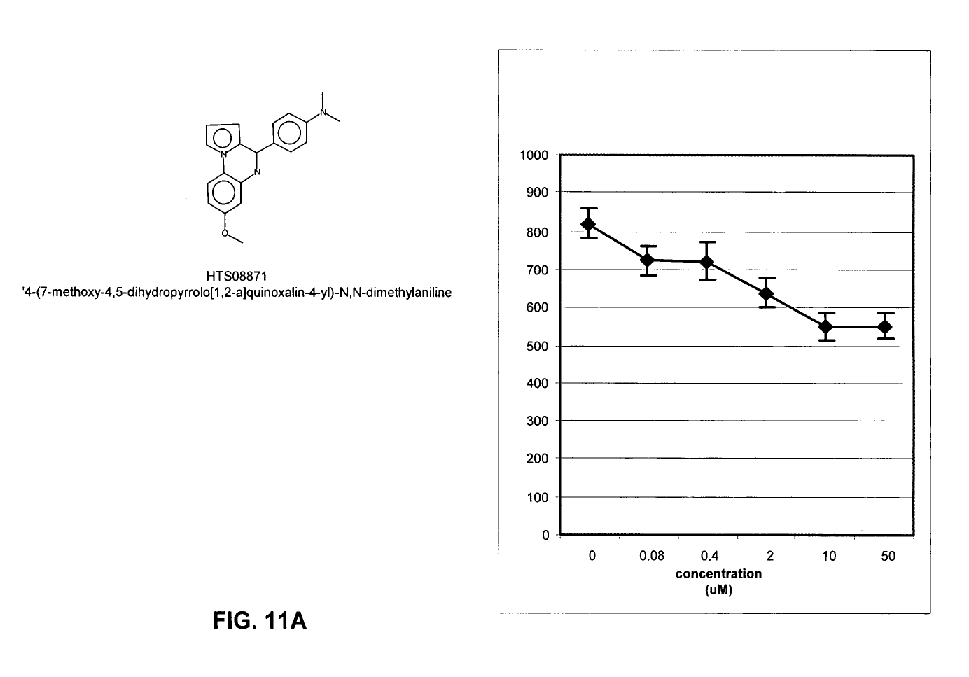

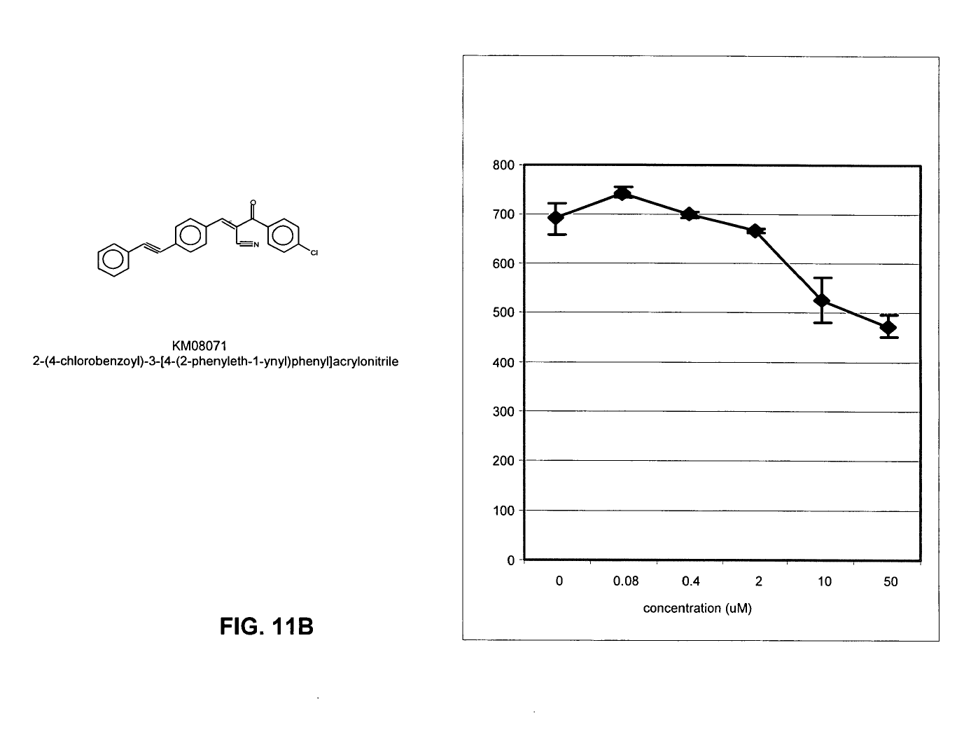

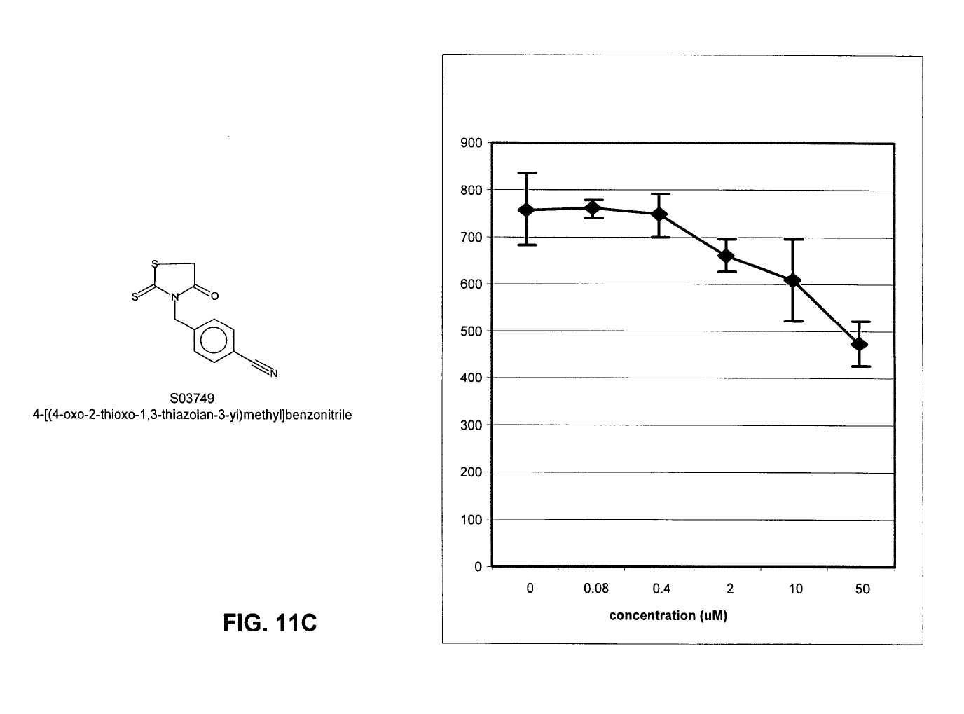

ELISA and DELFIA assays were then performed to evaluate the ability of the small molecules that were identified as “hits” in the AlphaScreens to inhibit the interaction of NgR and Nogo ligand. In the DELFIA Assay, 96 well streptavidin coated plates were blocked overnight with PBS and 10 mg/ml bovine serum albumin (BSA) (200 μl). The wells were then coated for 1.5 hours with sonicated HBH (Hanks Balanced Salt Solution/0.1 M HEPES/1 mg/ml BSA) containing Nogo 66 (B66) (0.5 μl of 10 mM/10 ml HBH) (50 μl) and then washed 4 times with 200 μl of HBH. The solution containing the inhibitor compound (50 μl in HBH) was added along with 1% FcNgR solution in HBH (50 μl), and the solution was incubated for 2 hours. The wells were washed 5 times with HBH. Alkaline phosphatase (AP) conjugated mouse anti-rat antibody (1:2500) was added and the wells were then washed 5 times with DELFIA wash buffer. The Europium (Eu) anti-mouse antibody was then added in Perlin Elmer Assay, buffer (100 μg/ml) (150 μl) and the wells were again washed 5 times with DELFIA wash buffer. The enhancer solution was added (100 μl) and the plate was read with the Perkin Elmer Victor 5 instrument 15 minutes later. (FIG. 10B) The results from the DELFIA assay indicate that compounds HTS08871, KM08071, and 503749, as well as the positive control, NEP33, inhibit the Nogo receptor:Nogo ligand (Nogo 66) interaction. (FIGS. 10D and 11A-C).

In the ELISA assay, 96 well streptavidin coated plates were blocked overnight with PBS and 10 mg/ml bovine serum albumin (BSA) (200 μl). The wells were then coated for 1.5 hours with sonicated HBH (Hanks Balanced Salt Solution/0.1 M HEPES/1 mg/ml BSA) containing Nogo 66 (B66) (0.5 μl of 10 mM/10 ml HBH) (50 μl) and then washed 4 times with 200 μl of HBH. The solution containing the inhibitor compound (50 μl in HBH) was added along with 1% FcNgR solution in HBH (50 μl), and the solution was incubated for 2 hours. The wells were washed 5 times with HBH. Alkaline phosphatase (AP) conjugated mouse anti-rat antibody (1:2500) was added and the wells were then washed 5 times with HBH. Colorimetric alkaline phosphatase substrate was added for 30 minutes and the plate was read with the Perkin Elmer Victor 5 instrument. (FIG. 10A). The results from the ELISA assay show that NEP33 and Cisplatin inhibit the Nogo receptor:Nogo ligand (Nogo 66) interaction. (FIG. 10C).

### Example 4

### Effect of Nogo Receptor on Neurite Outgrowth

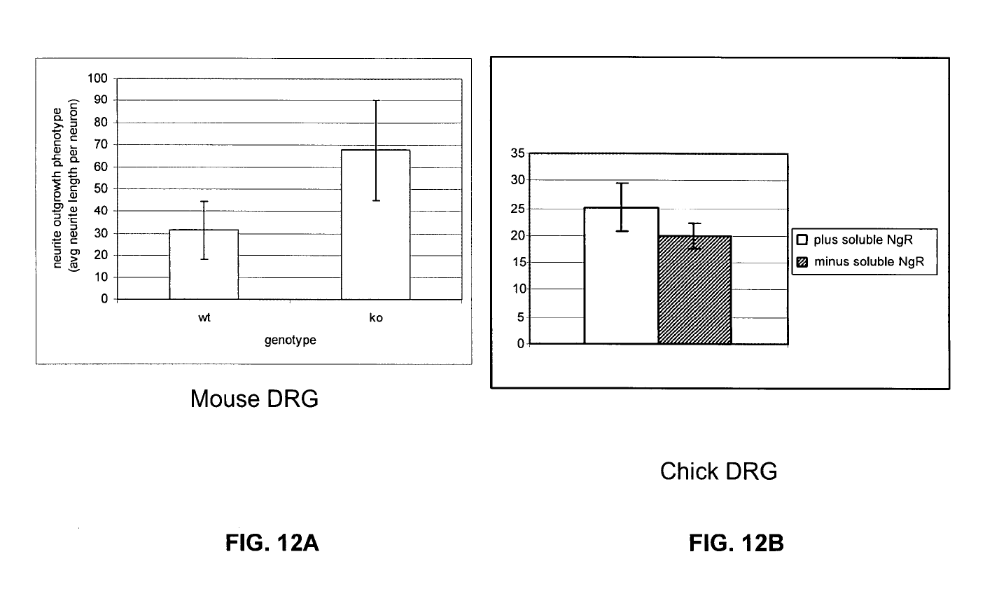

To demonstrate the effect of Nogo receptor on neurite outgrowth, Dorsal root ganglia (DRG) were removed from wild type and Nogo-receptor 1 (NgR1) knockout mouse pups at postnatal day 10, dissociated by trituration after 30 min incubation in 0.5% collagenase, plated on laminin-coated 96-well tissue culture plates in DMEM with 10% fetal bovine serum and B27. After 24 hours, cells were fixed in 4% formaldehyde in phosphate buffered saline (PBS), washed in PBS, and blocked for 1 hour in PBS with 0.1% triton X-100 and 10% goat serum, The cells were then incubated in rabbit anti-beta-3-tubulin (1:500) in PBS overnight. After washing 3 times, the cells were incubated in Alexa-fluor 488 goat anti-rabbit IgG (1:500) for 6 hrs, washed with PBS, and imaged at 10× with an ImagExpress automated microscope (Molecular Devices, Inc.). Neurite outgrowth was measured with AcuityExpress software (Molecular Devices, Inc.). These results indicate that Nogo receptor inhibits neurite outgrowth in the wild type mice. Neurite outgrowth is not affected in the Nogo receptor knockout mouse. (FIG. 12A).

### Example 5

### Neurite Outgrowth Assay

To test the ability of the “hit” compounds to promote neurite outgrowth, a neurite outgrowth assay was performed using each of these compounds. Dorsal root ganglia (DRG) were removed from chicken embryos at embryonic day 13 or 14, dissociated by trituration after 30 min incubation in 0.5% collagenase, and plated on laminin-coated 96-well tissue culture plates in DMEM with 10% fetal bovine serum containing 20 μM of the test compound. After two to four hours of incubation, the cells were fixed in 4% formaldehyde in phosphate buffered saline (PBS), washed in PBS, and blocked for 1 hour in PBS with 0.1% triton X-100 and 10% goat serum. The cells were incubated in rabbit anti-beta-3-tubulin (1:500) in PBS overnight and then washed 3 times with PBS. The cells were then incubated in Alexa-fluor 488 goat anti-rabbit IgG (1:500) for 6 hrs, washed with PBS, and imaged at 10× with an ImagExpress automated microscope (Molecular Devices, Inc.). Neurite outgrowth was measured with AcuityExpress software (Molecular Devices, Inc.).

The results indicated that 4′-(7-methoxy-4,5-dihydropyrrolo[1,2-a]quinoxalin-4-yl)-N,N-dimethylaniline (“HTS”), 2-(4-chlorobenzoyl)-3-[4-(2-phenyleth-1-ynyl)phenyl]acrylonitrile (“KM”), ethyl 5-[4-(dimethylamino)phenyl]-7-methyl-3-oxo-2,3-dihydro-5H-[1,3]thiazolo[3,2-a]pyrimidine-6-carboxylate (“555”), (4-chlorophenyl)(5-hydroxy-1-benzofuran-3-yl)methanone (“5470”), (4-chlorophenyl)(5-hydroxy-2-methyl-1-benzofuran-3-yl)methanone (“585”), 4-[(2-oxo-1,3-benzothiazol-3(2H)-yl)methyl]benzonitrile (“535”), 4-(1-benzoyl-1,2-dihydro-2-quinolinyl)-N,N-dimethylaniline (“536”), 3-(4-chlorobenzoyl)-6-methyl-4H-chromen-4-one (“5472”), 4-[(3-acetyl-7-ethyl-1H-indol-1-yl)methyl]benzonitrile (“794”) and N1,N1-dimethyl-4-[4-(dimethylamino)benzyl]aniline (“BTB11222”) promoted neurite outgrowth compared to the DMSO control. The results also indicated that 4-[(4-oxo-2-thioxo-1,3-thiazolan-3-yl)methyl]benzonitrile (“S”), 5-bromo-3-[2-(4-chlorophenyl)-2-oxoethyl]-3-hydroxy-1,3-dihydro-2H-indol-2-one (“5475”), 3-[2-(4-chlorophenyl)-2-oxoethyl]-3-hydroxy-1-methyl-1,3-dihydro-2H-indol-2-one (“592”), 3-[2-(4-chlorophenyl)-2-oxoethyl]-1-ethyl-3-hydroxy-1,3-dihydro-2H-indol-2-one (“664”) and 3-(4-chlorophenyl)-2-{2-[3-(2-methylprimidin-4-yl-phenyl]hydrazono}-3-oxopropanenitrile (“KMO2502”) inhibited neurite outgrowth compared to the DMSO control. (FIGS. 13, 15, 17A, 18A-C, 19A-B and 20).

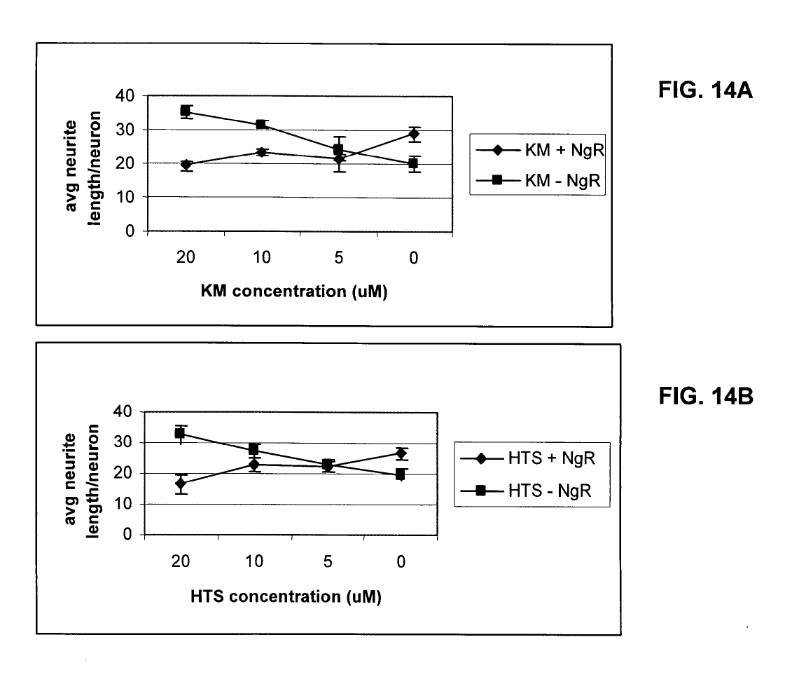

To further verify the mechanism of how the compounds are promoting neurite outgrowth, a competition assay was performed. First, the administration of 7.5 μM of a soluble Nogo receptor-Fc fusion protein (FcNgR) showed that FcNgR promotes neurite outgrowth. (FIG. 12B). compounds KM, HTS or 555 were then coadministered with 7.5 μM of FcNgR. These results showed that compounds KM, HTS, and 555 promoted neurite outgrowth in the absence of exogenous FcNgR, and that when the compounds and FcNgR are administered together in the same solution, the outgrowth promoting effects of both are inhibited. These results suggest that the compounds and exogenous FcNgR bind to each other, consequently mutually inhibiting their outgrowth-promoting effects. (FIGS. 14A-B and 17B). Thus, these results further suggest that the compounds are working by binding to NgR and inhibiting the interaction of NgR and Nogo ligand.

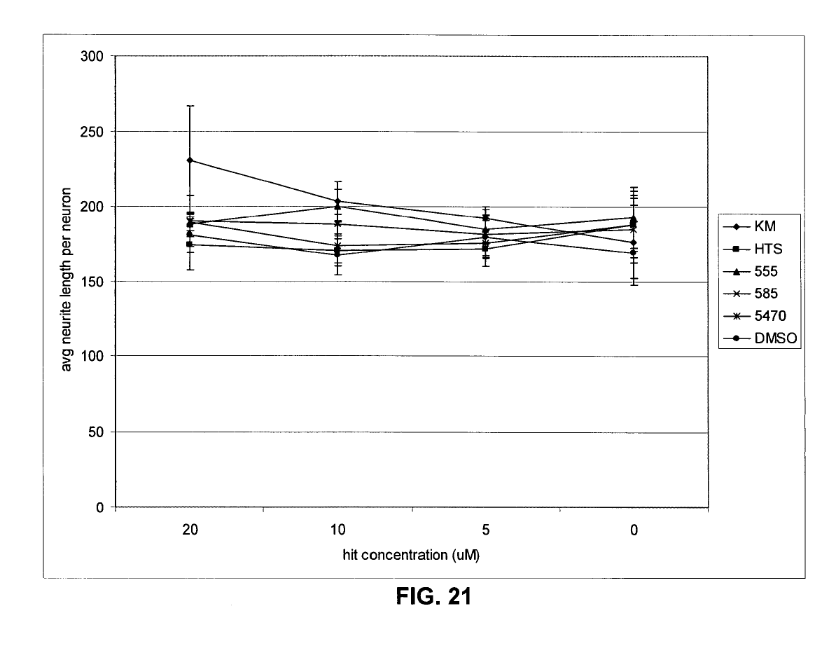

In another experiment to verify the mechanism of action, a neurite outgrowth assay was performed as described above except the Dorsal root ganglia (DRG) were removed from chicken embryos at embryonic day 8 instead of day 13. At day 8, Nogo receptor is not yet expressed in the DRG. The compounds KM, HTS, 555, 5470, 585 were administered as described above and neurite outgrowth was measured. The results showed that these compounds had no effect on the neurite outgrowth of day 8 DRGs suggesting that these compounds are working by binding to NgR and inhibiting the interaction of NgR and Nogo ligand. (FIG. 21)

However, the inhibition of neurite outgrowth by the compound S is believed to be independent of its interaction with the Nogo receptor-Nogo ligand complex. Dorsal root ganglia (DRG) were removed from wild type or NgR1 knockout mouse pups at postnatal day 15, dissociated by trituration after 30 min incubation in 0.5% collagenase, plated on laminin-coated 96-well tissue culture plates in DMEM with 10% fetal bovine serum and B27. After 24 hours, cells were fixed in 4% formaldehyde in phosphate buffered saline (PBS), washed in PBS, and blocked for 1 hour in PBS with 0.1% triton X-100 and 10% goat serum. The cells were then incubated in rabbit anti-beta-3-tubulin (1:500) in PBS overnight. After washing 3 times, the cells were incubated in Alexa-fluor 488 goat anti-rabbit IgG (1:500) for 6 hrs, washed with PBS, and imaged at 10× with an ImagExpress automated microscope (Molecular Devices, Inc.). Neurite outgrowth was measured with AcuityExpress software (Molecular Devices, Inc.). These results showed that the compound S inhibits neurite outgrowth in both wild type and NgR1 knockout mice, thus suggesting that the compound's effect on neurite outgrowth is independent from its interaction with the Nogo receptor-Nogo ligand complex.

As those skilled in the art will appreciate, numerous changes and modifications may be made to the preferred embodiments of the invention without departing from the spirit of the invention. It is intended that all such variations fall within the scope of the invention.

1. A method for identifying compounds which modulate the interaction of Nogo and Nogo receptor (NgR), comprising: (a) mixing a Nogo polypeptide, a NgR polypeptide and a test compound; (b) measuring an interference of the binding of said Nogo polypeptide to said NgR polypeptide in the presence of said compound, as compared to the binding of said Nogo polypeptide to said NgR polypeptide in the absence of said compound.

2-4. (canceled)

5. The method of claim 1, wherein said compound is a member of a small molecule library; wherein entire said small molecule library is screened according to the method of any one of claims 1-4; and wherein said small molecule library consists of drug-like organic compounds which have molecular weight of no more than 500 daltons, and have no more than 5 nitrogen or 5 oxygen atoms.

6-7. (canceled)

8. A method of claim 1, wherein said NgR polypeptide is Fc-NgR polypeptide.

9-11. (canceled)

12. A method of modulating the interaction of Nogo and Nogo receptor (NgR), comprising contacting said Nogo and Nogo receptor (NgR) with a compound, wherein said compound is an optionally substituted, optionally partially saturated benzofuran, indole, thiazolopyrimidine, pyrroloquinoxaline, benzothiazole, chromene or quinoline, or a salt thereof, and wherein said compound has a molecular weight of no more than 500 daltons, and has no more than 5 nitrogen or 5 oxygen atoms.

13. The method of claim 12, wherein said compound is selected from the group consisting of an optionally substituted 5-hydroxy-benzofuran, an optionally substituted 5-hydroxy-3-aroylalkylbenzofuran, an optionally substituted 3-acyl-indole, and an optionally substituted 3-hydroxy-3-aroylalkyl-1,3-dihydro-2H-indol-2-one or a salt thereof.

14. (canceled)

15. A method of modulating the interaction of Nogo and Nogo receptor (NgR), comprising contacting said Nogo and Nogo receptor (NgR) with a compound selected from the group consisting of 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, N1,N1-dimethyl-4-[4-(dimethylamino)benzyl]aniline, 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-l-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-{2-[3-(2-methylprimidin-4-yl-phenyl]hydrazono}-3-oxopropanenitrile or a salt thereof.

16. A method for identifying compounds which promote neurite outgrowth, the method comprising: (a) screening a small molecule library for compounds which interfere with the interaction of Nogo and Nogo receptor (NgR); (b) isolating a candidate compound, wherein said small molecule has a molecular weight of no more than 500 daltons; (c) conducting a secondary dose-response assay of said candidate compound, wherein said secondary dose-response assay is Enzyme-Linked ImmunoSorbent Assay (ELISA) or Dissociation-Enhanced Lanthanide Fluorescent Immunoassay (DELFIA); and (d) measuring neurite outgrowth activity of said candidate compound, wherein said neurite outgrowth is promoted in the presence of said candidate compound.

17-22. (canceled)

23. A method of promoting neurite outgrowth comprising contacting a neuron with an effective amount of a compound selected from the group consisting of 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline or a salt thereof.

24-28. (canceled)

29. The method of claim 31, wherein said compound is administered by oral, parenteral, subcutaneous, intravenous, intramuscular, intraperitoneal, transdermal, intracranial or buccal administration.

30. The method of claim 31, wherein said disease, disorder or injury is selected from the group consisting of multiple sclerosis, ALS, Huntington's disease, Alzheimer's disease, Parkinson's disease, diabetic neuropathy, stroke, traumatic brain injuries, spinal cord injury, optic neuritis, glaucoma, hearing loss, and adrenal leukodystrophy.

31. A method of treating a central nervous system (CNS) disease, disorder, or injury in a mammal, comprising administering to a mammal in need of treatment an effective amount of a compound selected from the group consisting of 4′-(7-methoxy-4,5-dihydropynolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3-yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline or a pharmaceutically acceptable salt thereof.

32-33. (canceled)

34. A method of promoting neurite outgrowth or axonal regeneration in a mammal comprising administering to a mammal in need thereof an effective amount of a compound selected from the group consisting of 4′-(7-methoxy-4,5-dihydropyrrolo[1,2-a]quinoxalin-4-yl)-N,N-dimethylaniline, 2-(4-chlorobenzoyl)-3-[4-(2-phenyleth-1-ynyl)phenyl]acrylonitrile, ethyl 5-[4-(dimethylamino)phenyl]-7-methyl-3-oxo-2,3-dihydro-5H-[1,3]thiazolo[3,2-a]pyrimidine-6-carboxylate, (4-chlorophenyl)(5-hydroxy-1-benzofuran-3-yl)methanone, (4-chlorophenyl)(5-hydroxy-2-methyl-1-benzofuran-3 -yl)methanone, 4-(1-benzoyl-1,2-dihydro-2-quinolinyl)-N,N-dimethylaniline, 4-[(2-oxo-1,3-benzothiazol-3(2H)-yl)methyl]benzonitrile, 4-[(3-acetyl-7-ethyl-1H-indol-1-yl)methyl]benzonitrile, 3-(4-chlorobenzoyl)-6-methyl-4H-chromen-4-one, and N1,N1-dimethyl-4-[4-(dimethylamino)benzyl]aniline or a pharmaceutically acceptable salt thereof.

35. A method for identifying compounds that inhibit neurite outgrowth, the method comprising: (a) screening a small molecule library for compounds which interfere with the interaction of Nogo and Nogo receptor (NgR); (b) isolating a candidate compound, wherein said small molecule has a molecular weight of no more than 500 daltons; (c) conducting a secondary response assay of said candidate compound, wherein said secondary dose-response assay is Enzyme-Linked ImmunoSorbent Assay (ELISA) or Dissociation-Enhanced Lanthanide Fluorescent Immunoassay (DELFIA); and (d) measuring neurite outgrowth activity of said candidate compound, wherein said neurite outgrowth is inhibited in the presence of said candidate compound.

36-41. (canceled)

42. A method of inhibiting neurite outgrowth or axonal regeneration comprising contacting a neuron with an effective amount of a compound selected from the group consisting of 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-{2-[3-(2-methylprimidin-4-yl-phenyl]hydrazono}-3-oxopropanenitrile or a salt thereof.

43. (canceled)

44. The method of claim 45, wherein said compound is administered by oral, parenteral, subcutaneous, intravenous, intramuscular, intraperitoneal, transdermal, intracranial or buccal administration.

45. A method of treating Schizophrenia or schizoaffective disorders, comprising administering to a mammal in need of treatment an effective amount of a compound selected from the group consisting of 4-[(4-oxo-2-thioxo-1,3-thiazolan-3-yl)methyl]benzonitrile, 5-bromo-3-[2-(4-chlorophenyl)-2-oxoethyl]-3-hydroxy-1,3-dihydro-2H-indol-2-one, 3-[2-(4-chlorophenyl)-2-oxoethyl]-3-hydroxy-1-methyl-1,3-dihydro-2H-indol-2-one, 3-[2-(4-chlorophenyl)-2-oxoethyl]-1-ethyl-3-hydroxy-1,3-dihydro-2H-indol-2-one, and 3-(4-chlorophenyl)-2-[2-[3-(2-methylprimidin-4-yl-phenyl]hydrazono}-3-oxopropanenitrile or a pharmaceutically acceptable salt thereof.

46. (canceled)
